SEQUENCE LISTING (5 sequences)

SEQ ID NO: 1 - protein, 10 aa, Artificial Sequence
  Synthetic peptide
VLYRYGSFSV

SEQ ID NO: 2 - protein, 9 aa, Artificial Sequence
  Synthetic peptide
NLVPMVATV

SEQ ID NO: 3 - protein, 6 aa, Artificial Sequence
  Synthetic peptide
QGPGAP

SEQ ID NO: 4 - DNA, 20 bp, Artificial Sequence
  Synthetic primer
aacccagcagacaatgtagc

SEQ ID NO: 5 - DNA, 22 bp, Artificial Sequence
  Synthetic primer
gtgtaacacgtctatacgccca

(19) United States
(12) Patent Application Publication
(10) Pub. No.: US 2014/0086828 A1
(43) Pub. Date: Mar. 27, 2014

(54) MODIFIED GOLD NANOPARTICLES FOR THERAPY

(71) Applicants: Aaron E. Foster, Houston, TX (US); Laura B. Carpin, Houston, TX (US); Adham S. Bear, Houston, TX (US); Rebekah Drezek, Spring, TX (US); Adam Yuh Lin, Houston, TX (US)

(72) Inventors: Aaron E. Foster, Houston, TX (US); Laura B. Carpin, Houston, TX (US); Adham S. Bear, Houston, TX (US); Rebekah Drezek, Spring, TX (US); Adam Yuh Lin, Houston, TX (US)

(21) Appl. No.: 13/700,582

(22) PCT Filed: May 31, 2011
(86) PCT No.: PCT/US11/38584

§ 371 Date: Dec. 3, 2013

Related U.S. Application Data
(60) Provisional application No. 61/349,319, filed on May 28, 2010.

(51) Int. Cl. A61K 47/48; A61K 49/00; A61K 51/12

(52) U.S. Cl. CPC A61K 47/48861; A61K 47/48238; A61K 47/48092; A61K 51/1244; A61K 49/0065; A61K 49/0067
USPC 424/1.17; 424/93.71; 424/93.21; 424/9.6; 424/489

(57) ABSTRACT

The present invention concerns methods and compositions utilizing gold nanoparticles for therapy for a medical condition, such as cancer. In particular aspects, the nanoparticles are included with or in a T cell to facilitate targeting of the nanoparticles to a desired location in vivo and/or to facilitate therapeutic treatment for the condition. In specific cases, the nanoparticle/T cell compositions further comprise microRNAs and/or peptides, and so forth.

### CROSS-REFERENCE TO RELATED APPLICATIONS

This application claims priority to U.S. Provisional Patent Application Ser. No. 61/349,319, filed May 28, 2010, which is incorporated by reference herein in its entirety.

### TECHNICAL FIELD

The present invention concerns methods and compositions utilizing gold nanoparticles for therapy for a medical condition. In particular aspects, the nanoparticles are utilized in compositions for therapeutic purposes.

### BACKGROUND OF THE INVENTION

Continuing research into cancer interventions has generated many innovative technologies. One such technology, gold nanoparticle-mediated photothermal therapy, has shown great success in animal studies (Dickerson et al., 2008; Hirsch et al., 2003; Melancon et al., 2008; O'Neal et al., 2004; Schwartz et al., 2009), and is currently undergoing clinical trials (see Nanospectra website). For therapy, gold nanoparticles (AuNPs) are systemically administered into the subject, and allowed to accumulate in the tumor via the enhanced permeability and retention (EPR) effect. The tumor is then irradiated with near-infrared (NIR) laser light, whose energy is absorbed by the AuNPs and translated into heat. Although delivery via the EPR effect has proven adequate for small animal studies, the larger scale of a human may present barriers to clinical translation.

A variety of particles have been employed for AuNP mediated-photothermal therapy, including silica-gold nanoshells (Hirsch et al., 2003), gold nanorods (Tong et al., 2007), gold nanocages (Chen et al., 2007), gold-gold sulfide nanoshells (Cole et al., 2009), and hollow gold nanoshells (Melancon et al., 2008). These particles are tuned to absorb in the NIR, which is the region of light maximally transmissive in tissue. Tumor accumulation of nanoparticles via the EPR effect is a result of tumor vasculature having a greater number of fenestrations than normal vasculature. The degree of accumulation is dependent on the shape of the AuNP chosen, as well as the hydrodynamic size and surface modifications of the particle, with peak tumor accumulations seen for polyethylene glycol (PEG)-coated particles with a hydrodynamic diameter of ˜60 nm (Perrault et al., 2009), Although silica-gold nanoshells are the most extensively researched of these particles for photothermal therapy applications, their large size (>100 nm) impairs in vivo delivery via the EPR effect. The addition of PEG to the nanoparticle surface, which is necessary to reduce reticuloendothelial uptake of the particles in vivo, further increases the hydrodynamic size. More recent studies have focused on particles such as the gold-gold sulfide and hollow gold nanoshells, which are smaller in size (20-40 nm). However, even with smaller particles, the percentage of the injected dose delivered to the tumor is usually very low, ranging from 1-10% of the injected dose (James et al., 2007; von Maltzahn et al., 2009), and delivery to non-target sites, such as the liver and spleen, is comparatively high (James et al., 2007; Nidome et al., 2006).

To address the delivery difficulties seen, a variety of nanoparticle surface modifications have been tried, including antibodies (Melancon et al., 2008) and hormone analogs (Lu et al., 2009). However, these modifications do not result in great improvements to the nanoparticle biodistribution or tumor accumulation. Alternative ideas, such as using silicon particle (Tasciotti et al., 2008) or macrophage (Choi et al., 2007) carriers to bear nanoparticles to the tumor site, have also been proposed, but to date no in vivo studies demonstrating the efficacy of such approaches have been performed.

### BRIEF SUMMARY OF THE INVENTION

The present invention is directed to methods and compositions encompassing nanoparticles for therapeutic purposes, including, for example, gold nanoparticles. In embodiments of the invention the compositions are employed for any individual in need thereof, but in specific embodiments the individual is in need of treatment for cancer. The cancer may be of any kind, but in specific embodiments the cancer is breast, lung, colon, skin, brain, prostate, pancreatic, spleen, gall bladder, cervical, endometrial, blood, bone, rectal, bladder, thyroid, esophageal, liver, kidney, and so forth. The cancer may be solid, but in specific embodiments the invention is utilized for non-solid cancer applications. The invention may be employed for any mammal, including human, dog, cat, horse, pig, sheep, goat, and so forth.

Embodiments of the invention include a composition comprising a gold nanoparticle and a biological moiety selected from the group consisting of T cell, peptide, and miRNA-targeting antisense nucleic acid. In some cases, the composition comprises at least one gold nanoparticle and at least one T cell. In particular cases, the composition comprises gold nanoparticles, T cells, and one or both of miRNA and antigenic peptide.

Nanoparticles of any kind may be utilized in the invention, so long as they can utilize T cells, peptides, and/or nucleic acids for delivery to a desired location in vivo. In specific embodiments gold nanoparticles are employed, although in some cases silver NP or gold-iron oxide NP (iron oxide coated in gold) may be used. The skilled artisan recognizes that the iron oxide NP is useful for MRI imaging. In certain cases, a mixture of nanoparticles are employed, either with the same biological entity (T cell, nucleic acid, and/or peptide) or with a different biological entity (T cell, nucleic acid, and/or peptide).

In some embodiments of the invention, the nanoparticles are separately conjugated to different therapeutics. In specific embodiments, a mixture is utilized for loading with T cells wherein some AuNPs have peptides (such as antigenic peptides) and/or some have nucleic acids (CpG, siRNA, miRNA inhibitors, or miRNA mimics, for example). In another embodiment, a single particle is conjugated with multiple therapeutics, layering it with both peptides and nucleic acids, then loading, for example. In specific embodiments, the nanoparticles further comprise drugs, including cancer drugs, for example. Exemplary cancer drugs include chemotherapeutics, hormone therapies, immunotherapies, and combinations thereof.

In some embodiments of the invention, there is a method for the in vivo delivery of a nanoparticle to a specific location within a subject comprising administering the nanoparticle-loaded T-cells to the subject. In some aspects the method further comprises contacting a plurality of nanoparticles with a plurality of T-cells. In certain embodiments, the specific location in the subject is a tumor, and in certain aspects the specific location in the subject is an organ. In particular cases, the T-cells specifically recognize at least one antigen including, for example, at least one tumor antigen. In particular embodiments, the T-cells are genetically modified to specifically recognize at least one tumor antigen. In some aspects, the nanoparticle is a gold nanoparticle.

In particular embodiments, the T cell provides a therapeutic effect as part of the gold nanoparticle composition. However, in some cases, the gold nanoparticle also provides a therapeutic effect, for example in the event that the nanoparticle may be heated when it is localized to areas in vivo that are accessible to a heat-producing device, such as a laser. Exemplary cases may include breast or skin cancer, for example.

In specific cases, the T cell is modified to enhance the localization capabilities and/or therapeutic capabilities of the composition. In specific cases, the T cell is modified to enhance function or biodistribution, for example. In specific embodiments, the T cell comprises one or more tumor markers. In some aspects, the T cells encompass engineered tumor-specific or chemokine receptor-modified T cells.

In some embodiments of the invention, there is a method of treating cancer in an individual, comprising the step of administering to the individual an effective amount of one or more compositions comprising one or more gold nanoparticles and a T cell. In some cases, the one or more gold nanoparticles are comprised within the T cell. In specific embodiments, the composition localizes to a location in vivo that is accessible to heat and heat is applied thereto.

In some aspects, the composition further comprises one or more peptides, and in some cases the peptide is conjugated to the nanoparticle. In specific aspects, the conjugation of the peptide to the nanoparticle is pH-sensitive. In specific embodiments, the peptide is from 15-40 amino acids in length. The peptide may be further defined as an antigenic peptide. The one or more peptides may be all the same peptides, or the one or more peptides may include different peptides. In cases wherein the peptide is conjugated to the nanoparticle there may be one or more peptides attached in tandem thereto.

In particular cases, the composition further comprises one or more nucleic acids, and the nucleic acids may be conjugated to the nanoparticle. In specific cases, the conjugation of the nucleic acid to the nanoparticle is pH-sensitive. The nucleic acid may be an antisense nucleic acid, for example an antisense oligonucleotide that targets miRNA, such as an antisense oligonucleotide that targets miRNA comprises miRNA mimics, in some cases. In particular embodiments, the nucleic acid comprises locked nucleic acid. In some embodiments, the nucleic acids are no longer than 25 nucleotides in length. Although the miRNA may be any kind, in specific cases the miRNA comprises mir-221, mir223, mir155, mir-21, mir-138, mir-181 mir-122, or mir-765.

In some embodiments of the invention, the method further comprises administering to the individual an effective amount of one or more compositions comprising one or more gold nanoparticles and a biological moiety selected from the group consisting of a peptide, a nucleic acid, and both peptide and nucleic acid.

In particular aspects, the T cell recognizes at least one tumor antigen, for example, survivin, telomerase, MAGEA1, MAGEA3, MART1, RHAMM, gp100, ROR1, PRAME, SSX2, SSX4, RONIN, Her2Neu, PSMA, PSCA, or HMW-MAA. In specific cases, the T cell recognizes any protein that is aberrantly expressed or over expressed compared to normal tissue.

The T cell may be modified, in certain aspects, and such modification may be genetic or non-genetic. For example, a genetically modified T cell may be further defined as expressing a chemokine receptor, a tumor antigen, a chimeric antigen receptor, therapeutic molecule included immune modulating cytokines (e.g. IL-12) or proteins (e.g. Flagellin, CD40L). In specific embodiments, the chemokine receptor is selected from the group consisting of CCR7, CCR2, CXCR4, CXCR1, and CXCR2. The chimeric antigen receptor may comprise wherein the chimeric antigen receptor comprises scFv: GD2, CD19, Her2neu, PSMA, MUC1, SSEA1, CEA; and/or ligands: Wnt5a. In some cases, the modified T cell expresses CCR7, adhesion molecule CD62L, or both.

In aspects wherein the modified T cell is further defined as being non-genetically modified, such modification may be exposure to culture comprising one or more cytokines, such as a cytokine selected from the group consisting of IL-7, IL-15, IL-12, or IL-21.

In some aspects, the composition is administered to the individual more than once.

In particular embodiments, the composition further comprises a detectable marker, such as fluorescence, magnetism, or radioisotope, for example.

The shape of the nanoparticle may be round, elliptical, rod-shaped, wire-shaped, prism-shaped, or is a quantum dot. The width of the nanoparticle may be from 5 nm to 1000 nm, 5 nm to 200 nm, 40 nm to 100 nm, and so forth.

In some cases, the composition further comprises polyethylene glycol (PEG).

The composition may be administered by any suitable route including subcutaneously, intravenously, intraperitoneally, or intradermally.

In embodiments of the invention, there are one or more composition comprising one or more gold nanoparticles and a T cell. The compositions may further comprise one or more peptides, one or more nucleic acids, or both.

In some cases, there is a kit comprising a composition of the invention, said composition housed in a suitable container.

In certain embodiments, a peptide-loaded nanoparticle is employed as a vaccine composition. For example, there are compositions of and methods for vaccination against an infectious agent using a peptide-loaded nanoparticle composition for elicitation of an immune response in a mammal. In specific embodiments, there are vaccines against malaria using nanoparticles loaded with peptides to the P. yeolii circumsporozoite antigen, for example. Other infectious agents in which similar approaches may be employed include a microbe or microorganism such as a virus, bacterium, prion, or fungus. Pathogenic viruses include those of the families of Adenoviridae, Picornaviridae, Herpesviridae, Hepadnaviridae, Flaviviridae, Retroviridae, Orthomyxoviridae, Paramyxoviridae, Papovaviridae, Polyomavirus, Rhabdoviridae, or Togaviridae. Pathogenic bacteria include Mycobacterium tuberculosis, Streptococcus Pseudomonas, Shigella, Campylobacter and Salmonella and the pathogenic bacteria that cause infections such as tetanus, typhoid fever, diphtheria, and syphilis. Specific examples include Mycobacterium leprae, Yersinia pestis, Rickettsia prowazekii, Bartonella spp., Streptococcus pneumonia, and Spanish influenza virus

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1. Gold nanoparticle uptake by human cytotoxic T cells. a) TEM imaging of gold colloid (diameter=40-45 nm) b) Darkfield images of human T cells demonstrate gold nanoparticle uptake by the increased scattering seen in the AuNP group c) TEM images of AuNP-T cells with gold nanoparticles visualized intracellularly. No particles are present in the nucleus. d) ICP-OES analysis of T cell intracellular gold content at 12 hours using different loading concentrations. Each point represents data acquired from three different T cell donors. e) Time course data for T cells loaded with different concentrations of gold nanoparticles. The highest loading concentrations show the same number of AuNPs per cell at 24 hours, indicating intracellular saturation of gold particles.

FIG. 2. AuNP loading has no effect on T cell viability or function. T cells were loaded in the presence of 0.5 nM AuNPs for 24 hrs. (a) T cell viability was assessed using FACS by surface staining for Annexin V and 7-MD. Cells negative for both markers were considered viable. (b) T cell proliferation was measured by 24 hr [³H] thymidine incorporation. (c) T cell migratory potential was determined by performing an in vitro transwell migration assay toward LCL supernatant. (d) IFN-□ secretion following PMA-I stimulation was determined by FACS. Values are average ±SEM.

FIG. 3. AuNP-loaded T cells migrate to tumors in vivo. a) Bioluminescent imaging of AuNP-T cell biodistribution at 48 hours (n=3). T cell localization can be seen at the tumor site (circle) and within the spleen. b) Microscopy of tumor tissue sections. Top column shows immunohistochemistry for CD3 expression, a marker of T cells. Bottom column shows correlating darkfield images of the same field. Areas of increased scattering within the darkfield images, which indicate the presence of gold nanoparticles, correlate well with CD3 positivity.

FIG. 4. Biodistribution of PEGylated AuNPs and AuNP-loaded human T cells in mice. AuNP-T cells accumulate in the lung, liver, spleen, and bone. Values are percentage of the injected gold dose normalized by the dry weight of the collected tissue ±SEM. For the lungs, liver, and bone, the AuNP-T cells had higher percentages of gold than the AuNP for all possible comparison pairs by one-way ANOVA and Tukey's test (p<0.05). The AuNP group had a higher percentage of gold than the AuNP-T cell group for all possible comparison pairs for the intestine by one-way ANOVA and Tukey's test (p<0.05). No significant differences were seen between the treatment groups for spleen, kidney, muscle, heart, or brain. AuNPT cell biodistribution correlates well with the predicted biodistribution of human T cells within a mouse model.

FIG. 5. T cells more efficiently deliver AuNPs to the tumor site. a) Time points of 24 hours for the AuNP group and 48 hours for the T cell group were selected based on optimal tumor gold accumulation. b) The percentage of gold delivered to the tumor by the T cells is greater than the amount of gold delivered to the tumor using systemic injection of PEGylated AuNPs by student's t-test (n=8 mice for AuNP, n=11 mice for AuNP-T cell, P<0.01). Percentages shown are normalized by dry tumor weight, and are the mean±SEM.

FIG. 6. a) Gold colloid size distribution. b) Gold colloid absorbance spectra.

FIG. 7. T cell phenotype post AuNP Loading. Phenotyping indicates that the presence of gold nanoparticles does not affect T cell differentiation. Gold nanoparticle internalization is not preferentially toxic to a specific T cell population.

FIG. 8. Normal biodistribution of human T cells in a tumor-bearing mouse model. T cells were retrovirally transduced with firefly luciferase and injected into mice (n=3). There is notable T cell accumulation in the lung and tumor at 24 hours post injection. By 72 hours, T cells have migrated out of the lungs and are predominantly seen in the tumor. The inventors also have data that shows a 48 hour timepoint and that demonstrates clearer spleen accumulation. Here, the spleen accumulation is not seen due to the brightness of the tumor signal.

FIG. 9. Design of a CSP-NV. P. yoelii CSP antigen structure contains a CD4+ and CD8+ T cell epitope as well as the central repeat region which stimulates CSP-specific antibody production. This protein sequence can be used to generate an overlapping peptide library that will subsequently be conjugated to gold nanoparticles (AuNP) using PEG and EDC chemistry, for example.

FIG. 10. In vitro stimulation of antigen-specific T cells using gp100-NV. A) Co-culture of gp100-NV with murine bone marrow derived DC shows that NV are efficiently taken up into the APCs. B and c) DC pulsed with gp100-NV were subsequently used to stimulate pmel-1 T cells, which recognize gp100 peptide antigen, and measured by IFN-□ ELIspot. Gp100-NV demonstrated comparable stimulation to free-peptide indicating delivery is feasible.

FIG. 11. Conjugation of an antigen peptide pool to AuNP. a) and b) Conjugation of peptides to AuNP in the presence of excess linker allows a time-dependent growth of NV. c) and d) Design of a peptide-pool conjugated NV either with a single peptide or with multiple peptide antigens indicates that delivery of a multivalent stimulus is feasible.

FIG. 12. Synthesis of AuNP and conjugation to LNAs targeting the exemplary miR-221. a) Monodispersed AuNPs of 40 nm are synthesized by CO reduction. b) Uptake of AuNPs by T lymphocytes using darkfield microscopy. c) Thiol-modified oligo nucleotides are subsequently bound to AuNPs using DTT, creating an anti-miR targeting conjugate.

FIG. 13. Reporter assay for testing AuNP-LNA inhibition of miR-221 in B-CLL. a) ˜400 bp of the p27 3′ UTR was cloned into pmiR-GLO. The corresponding antisense miR-221 sequence and LNA sequence are shown. b) Transfection of miR-221-expressing HELA cells suppresses FFluc expression, but treatment with anti-miR-221 LNA restores FFluc (relative Luc) expression.

FIG. 14. Exemplary formation of pH-sensitive hydrazone bonds.

FIG. 15. Exemplary schematic of loss of moieties upon a drop in pH.

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially of” or “consist of” one or more sequences of the invention, for example. Some embodiments of the invention may consist of or consist essentially of one or more elements, method steps, and/or methods of the invention. It is contemplated that any method or composition described herein can be implemented with respect to any other method or composition described herein.

### I. DEFINITIONS

The term “nanoparticle” as used herein generally refers to a particle <100 nm, although in some embodiments it refers to particles <1000 nm. The particles of the invention in specific embodiments are <100 nm, but with some peptide conjugations they can grow to larger than 300 nm.

The term “T cell” (which may also be referred to in the art as a “T lymphocyte” as used herein refers to a lymphocyte from the thymus that is able to recognize specific antigens and can activate or deactivate other immune cells.

### II. GENERAL EMBODIMENTS

In general embodiments of the invention, a composition is localized to a desired location in vivo because of inherent properties for one or more of the moieties of the composition. In specific embodiments of the invention, the composition comprises Au nanoparticles that are capable of localizing to tumor vasculature and also comprises a T cell that is capable of localizing within tumor sites, for example in response to tumor-associated signals and/or environment.

The exemplary bipartite composition may further be comprised of additional moieties that are capable of enhancing localization to a specific site in vivo and/or that are capable of providing an indirect or direct therapeutic purpose. In specific embodiments, the additional moieties include peptides, nucleic acids, and/or drugs. The peptides may be antigenic peptides, in specific embodiments. The nucleic acids may be antisense nucleic acids, including antisense oligonucleotides. The antisense nucleic acids can be antisense to a mRNA or a miRNA, in some cases. In cases wherein the nucleic acid is antisense to a miRNA, the entity may be referred to as an antagomiR. The target miRNA may be of any kind so long as the composition has access to the miRNA and at least some of the sequence of the antisense nucleic acid is sufficient to hybridize to at least some of the miRNA.

In certain embodiments, the compositions are labeled such that the may be employed for diagnostic imaging, including imaging of a tumor. The compositions in some cases may be labeled with any suitable label so long as it is informative of the in vivo location of the tumor. The label in specific cases may be fluorescent, magnetic, radioisotopes, and so forth. In specific embodiments the compositions are used for both therapeutic and diagnostic purposes.

In particular embodiments, the compositions or part thereof are modified with polyethylene glycol (PEG). The skilled artisan recognizes that PEG is employed to improve circulatory times so as not to be sequestered by the liver, for example. In embodiments wherein nucleic acids and/or peptides are employed, the PEG may be utilized as a linker to AuNP.

In some cases, the composition of the invention is administered to the individual once, although in some cases the composition is administered to the individual more than once, including, for example, within hours, days, weeks, months, or years of the initial administration. In some embodiments, the compositions are administered multiple times in one week, weekly, bi-weekly, or monthly.

In certain aspects of the invention, one or more compositions of the invention are administered to an individual in need of vaccination.

### III. NANOPARTICLES

The invention employs nanoparticles in conjunction with T cells and, optionally, other biological moieties, to localize a composition to a desired location in vivo, including at least one cancer cell. The nanoparticles may be of any kind so long as they are capable of being localized with a T cell to a cancer cell, but in a specific embodiment the nanoparticle is a gold nanoparticle. Alternative nanoparticles include silver nanoparticles or gold-iron oxide nanoparticles (iron oxide coated in gold).

In specific embodiments the nanoparticle is generally spherically, including round, although it may be elliptical, rod-shaped, wire-shaped (in the art considered longer than a rod), prism-shaped, and so forth. In specific embodiments the nanoparticles are quantum dots. The skilled artisan recognizes that the size of the nanoparticle is considered according to its application. For example, in an effort to avoid the entire composition becoming too large, one can reduce the size of the nanoparticle if the accompanying biological moiety is large, and vice versa. In some cases, the size of the nanoparticle is between 5 nm and 1000 nm in width, between 5 nm and 400 nm, between 5 nm and 200 nm, between 5 nm and 100 nm, between 5 nm and 50 nm, and so forth. In particular aspects, the nanoparticle is less than 100 nm. In specific cases, the nanoparticle is 40-45 nm.

### IV. T CELL DELIVERY OF NANOPARTICLES

T cells have the ability to migrate throughout the body and accumulate within tumor sites in response to tumor-associated chemokines. This unique tumor-tropic property permits their use as cellular vehicles for the delivery of molecular therapeutics (Cole et al., 2005; Harrington et al., 2002; Qjao et al., 2008; Yotnda et al., 2004). In addition, T cells are easily isolated from peripheral blood and can be expanded to large numbers ex vivo, unlike other potential tumor-tropic cellular vehicles, such as macrophages or mesenchymal stromal cells. T cells may also be genetically modified to enhance their function or biodistribution in vivo (Di Stasi et al., 2009; Kershaw et al., 2002). Combining the advantages of T cells with nanotechnology is useful to generate innovative new approaches to cancer therapy. Previous studies have used iron-oxide nanoparticles for antigen-specific T cell sorting (Gunn et al., 2008) and in vitro imaging (Flynn et al., 2007) Thus, in specific embodiments of the present invention, T cells are used as AuNP carriers to increase gold delivery to tumor sites in vivo.

In some embodiments of the invention, a modified T cell and gold nanoparticle are employed in conjunction to treat cancer. In specific cases, the T cell is modified to enhance the therapeutic function and/or localization, for example. In specific embodiments, the T cell comprises one or more tumor markers. In some aspects, the T cells encompass engineered tumor-specific or chemokine receptor-modified T cells.

The modified T cells may be modified genetically and/or non-genetically. Genetic modification includes modifying the T cell to express a particular surface antigen, for example, or other therapeutic molecules (e.g. cytokines, chemokines, immune modulators and cytotoxic molecules). The T cells in exemplary cases may express chemokine receptors that facilitate where the T cell composition localizes. Examples of chemokine receptors include CCR7, CCR4, and CCR2. In other cases, the T cells are modified in a non-genetic manner, such as by culturing the T cells with compounds that facilitate their therapeutic and/or localization capabilities. In specific cases, one can culture the T cells with cytokines that reprograms the T cells to become central memory T cells, and examples include IL-7, IL-15, IL-21, and so forth.

The T cell may be modified to express CCR7 that recognizes CCL19 and CCL21. The T cell may be modified to express CD62L adhesion molecule that allows the composition to bind to lymph organs. T cells may also be engineered to express chimeric antigen receptors (CARs). The CARs comprise a binding moiety specifically recognizing a tumor cell surface antigen and a lymphocyte-activating signaling chain, in some cases, and the CAR-mediated recognition induces cytokine production and tumor-directed cytotoxicity of T cells. Further embodiments of CARs include signal sequences from various costimulatory molecules, resulting in enhanced T-cell persistence and sustained antitumor reaction. In specific cases the CARs are fusions of single-chain variable fragments (scFv) derived from monoclonal antibodies or natural or truncated ligands fused to CD3-zeta transmembrane and endodomain. (scFv: GD2, CD19, Her2neu, PSMA, MUC1, SSEA1, CEA; ligands: Wnt5a, for example). Genetic modification with T cell receptor alpha and beta chains isolated from antigen-specific T cell clones may also be used.

Tumor antigens may be present on the surface of the T cell, for example upon pulsing the T cells with tumor antigen peptides that is well known in the art. In some cases, the tumor antigen is expressed as a transgene in the T cell, for example.

In specific embodiments, any combination of molecules that can improve or change immune function may be used in the invention. In embodiments of the T cell vaccine subject matter, T cells may be employed that are modified with one or more of the following: 1) chemokine receptor for directing where they go in vivo (CCR7 for lymph node homing, for example); 2) therapeutic molecule to target and stimulate immune cells (for example, CD40L, IL-12 or flagellin to stimulate B cells and dendritic cells) in vivo; and 3) tumor antigen, expressed as a transgene or pulsed onto the cells as a peptide.

### V. NANOPARTICLE COMPOSITIONS COMPRISING PEPTIDES

In certain embodiments of the invention, there is a composition comprising a nanoparticle and one or more peptides. In specific cases, the composition also comprises a T cell. The peptides may be further defined as antigenic peptides. In specific embodiments, the peptides are conjugated to the nanoparticle. The nanoparticle may be coated with one or more peptides, and in some cases the nanoparticle is coated with a monolayer of peptides or multiple layers having peptides attached in tandem. The peptides may be all the same peptide or may be different peptides. In some cases, the peptide provides an epitope or is a peptide for cytotoxic T cells and helper cells.

The peptides may be of any length so long as the nanoparticle maintains a suitable size. In some cases, the peptides are between 15 and 40 amino acids in length, between 15 and 35 amino acids in length, between 15 and 30 amino acids in length, between 15 and 25 amino acids in length, or between 15 and 20 amino acids in length, for example. The peptides may be 15, 16, 17, 18, 19, 20, 21, 22, 23, 24, 25, 26, 27, 28, 29, 30, 31, 32, 33, 34, 35, 36, 37, 38, 39, or 40 amino acids in length, for example.

The T cells may be exposed to a library of peptides the plurality of which cover part of or an entire polypeptide. In such a case, the peptides may overlap a certain number of amino acids, including, for example, an overlap of 5-15 amino acids, depending on the length of the peptide. The overlap may be 11 amino acids, less than 9 amino acids, 15 amino acids, and so forth.

In some cases of the invention, the composition employs peptides that are deposited in the lymph glands and/or spleen.

### VI. NANOPARTICLE COMPOSITIONS COMPRISING NUCLEIC ACIDS

In certain embodiments of the invention, there are nanoparticle compositions comprising one or more nucleic acids. The compositions may further comprise a T cell, in certain aspects. The nucleic acids are conjugated to the nanoparticle, in certain instances. In some cases, the nucleic acids comprise DNA, RNA, or LNA, for example. RNA duplexes, miRNA mimics, siRNA, and so forth may be employed. In particular embodiments the nucleic acids provide a therapeutic benefit, for example being capable of hybridizing in an antisense manner to a polynucleotide with deleterious effects or to a polynucleotide that expresses a gene produce, including a protein, with deleterious effects.

In specific embodiments, the nucleic acid is an antisense oligonucleotide. The nucleic acid may be of any suitable length so long as the composition comprising the nanoparticle is not excessive in size. In specific cases, the nucleic acid is not longer than 25, 24, 23, 22, 21, 20, 19, 18, 17, 16, 15, 14, 13, 12, 11, 10, 9, or 8 nucleotides, for example. In some cases, the nucleic acid further comprises polyA or polyT, for example as a spacer (although PEG may be alternatively used as a spacer).

Exemplary miRNAs include at least let-7, let-7A, let-7C, let-7F-2, miR-7, miR-9a, miR-9-as, miR-10a, miR-15A, miR-16, miR-17, miR-20, miR-21, miR-22, miR-23a, miR-23b, miR-24, miR-25, miR-26a, miR-28, miR-29, miR-29b, miR-30a, miR-30a-as, miR-31, miR-92, miR-95, miR-99b, miR-103, miR-105, miR-106a, miR-125a, miR-126, miR-126-as, miR-130a, miR-133, miR-135a, miR-137, miR-138, miR-139, miR-140, miR-141, miR-143, miR-144, miR-145, miR-152, miR-155, miR-181a, miR-181b, miR-182, miR-183, miR-186, miR-188, miR-189, miR-192, miR-194, miR-195, miR-199a, miR-199a-as, miR-200b, miR-201, miR-203, miR-205, miR-211, miR-215, miR-219, miR-221, miR-222, miR-223, miR-224, miR-290, miR-291, miR-291-5P, miR-298, miR-301, miR-326, miR-328, mu-miR-329, miR-331, miR-341, miR-342, miR-344, miR-361, and miR-425.

### VII. PHARMACEUTICAL PREPARATIONS

Pharmaceutical compositions of the present invention comprise an effective amount of one or more compositions comprising nanoparticles and T cells (and/or nucleic acids and peptides) or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one composition or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The composition may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The composition may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include the composition, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the composition may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In preferred embodiments of the present invention, the compositions are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution).

Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, composition may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the composition may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### VIII. COMBINATION TREATMENTS

In order to increase the effectiveness of a composition of the invention, it may be desirable to combine these compositions with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with other therapies.

Alternatively, the additional therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other, or greater. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, gene therapy is “A” and the secondary agent, such as radio- or chemotherapy, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the compositions of the present invention to a patient may follow general protocols for the administration of chemotherapeutics. It is expected that the treatment cycles would be repeated as necessary. It also is contemplated that various standard therapies, as well as surgical intervention, may be applied in combination with the described hyperproliferative cell therapy. Exemplary additional therapies include chemotherapy, hormonal therapy, immunotherapy, surgery, radiation, and so forth.

### IX. EMBODIMENTS OF KITS OF THE INVENTION

Any of the compositions described herein may be comprised in a kit. In a non-limiting example, a nanoparticle, including a gold nanoparticle, comprised with a T cell and, optionally, a peptide or nucleic acid, may be comprised in a kit. In alternative embodiments, the gold nanoparticle is comprised with a peptide in the absence of a T cell and/or with a nucleic acid in the absence of a T cell.

The kits may comprise a suitably aliquoted composition of the present invention. In some cases, the kit comprises separate components of the composition and/or reagents suitable to assemble the components and or modify them, including linkers, for example. The components of the kits may be packaged either in aqueous media or in lyophilized form. The container means of the kits may generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the composition and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

Irrespective of the number and/or type of containers, the kits of the invention may also comprise, and/or be packaged with, an instrument for assisting with the injection/administration and/or placement of the ultimate composition within the body of an animal. Such an instrument may be a syringe, pipette, forceps, and/or any such medically approved delivery vehicle, for example.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### T Cell Delivery of Gold Nanoshells for Cancer Immunotherapy

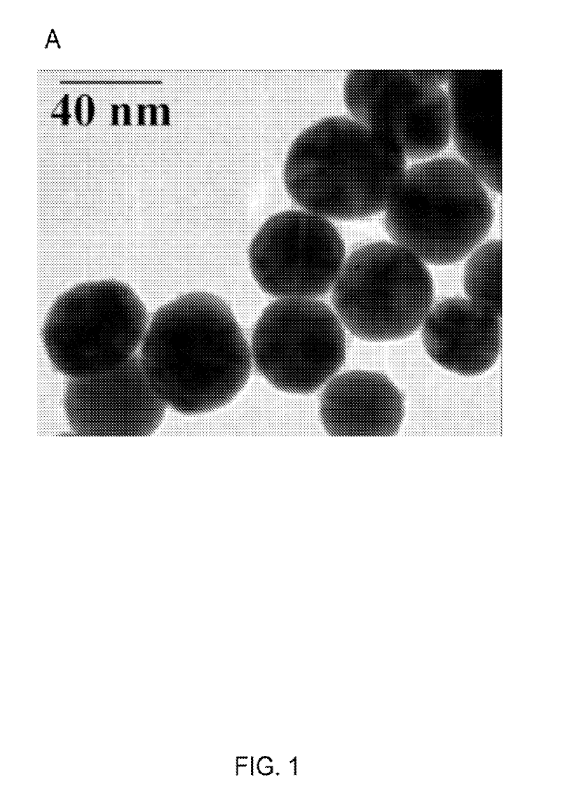

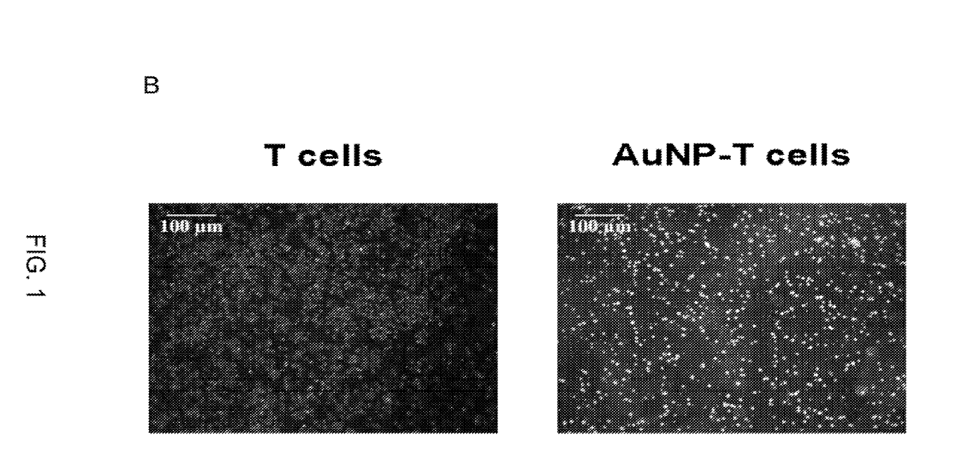

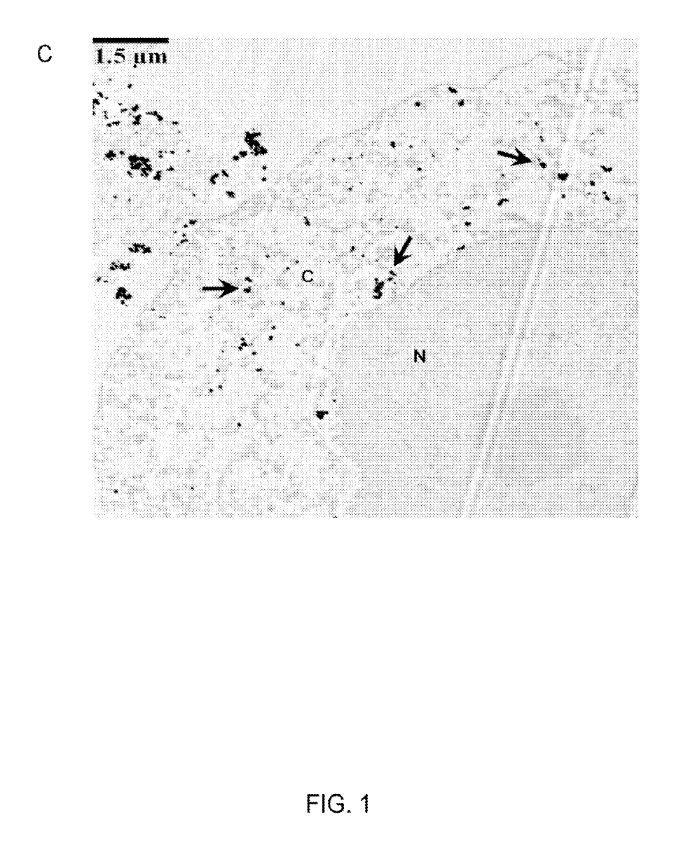

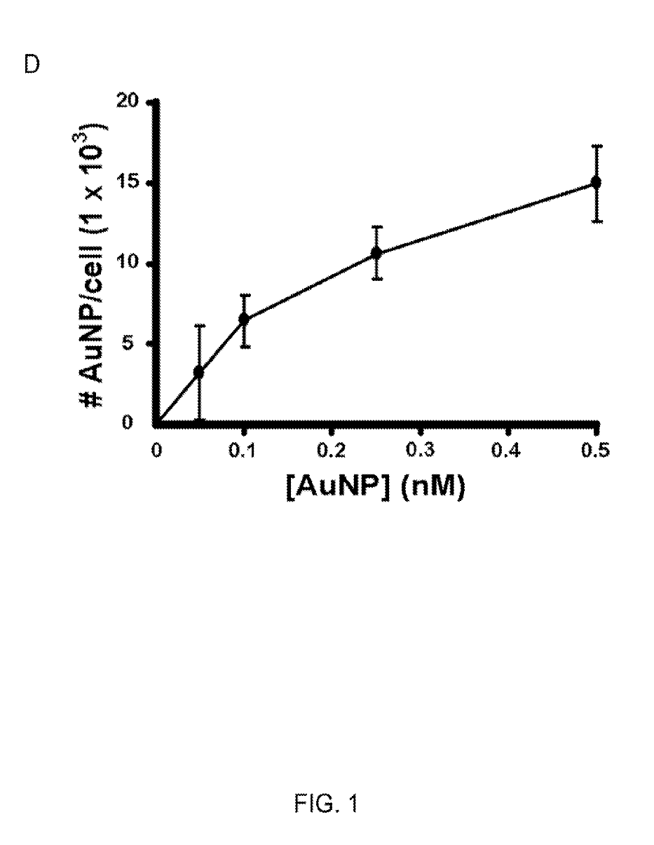

For this Example, where therapeutic optical properties were not needed, gold colloidal nanospheres were employed, which can be easily synthesized at the optimal size for intracellular uptake. The synthesized gold colloid was 40-45 nm in diameter by transmission electron microscopy (TEM) (FIG. 1a), with an absorbance peak of 529 nm (FIGS. 6a & 6b). Human T cells were isolated from the peripheral blood of consenting donors. T cells were cultured in the presence of gold colloid for a period of 1-24 hours to permit AuNP internalization. AuNP loading was confirmed using darkfield microscopy (FIG. 1b). T cells incubated with AuNPs demonstrated increased scattering under darkfield, suggesting internalization. TEM of the AuNP-T cells further confirmed internalization (FIG. 1c), with AuNP aggregates seen within the cytosol of the T cells. To optimize T cell loading, the inventors varied initial AuNP loading concentrations and incubation duration. By altering these variables, they were able to modulate the number of nanoparticles per T cell. A maximum of 14900±2400 AuNPs was internalized per T cell when cultured in the presence of 0.5 nM AuNP concentration for 12 hours (FIG. 1d), and T cells became saturated at this concentration within 24 hours (FIG. 1e). For each of the remaining studies, T cells were incubated with 0.5 nM of the gold colloid for 24 hours to maximize AuNP-T cell gold content.

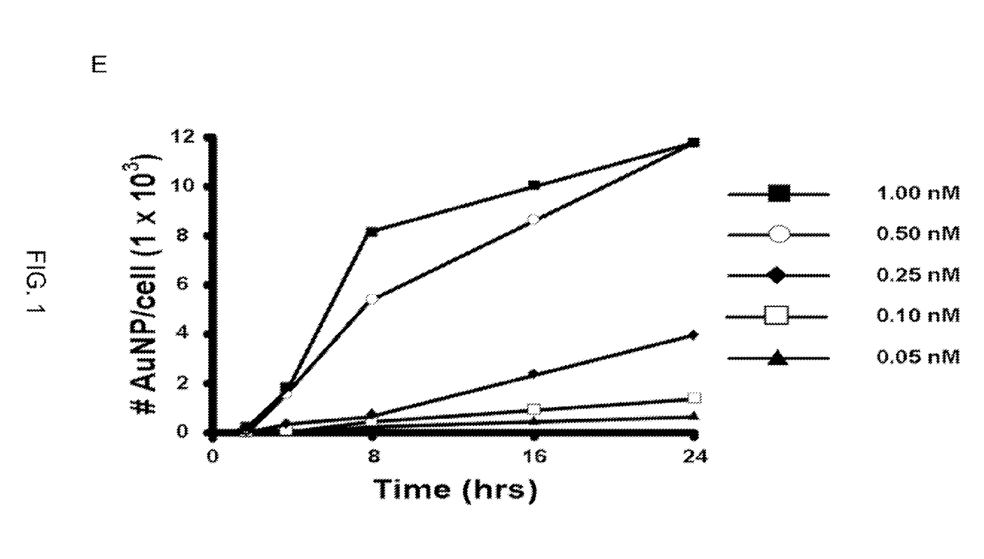

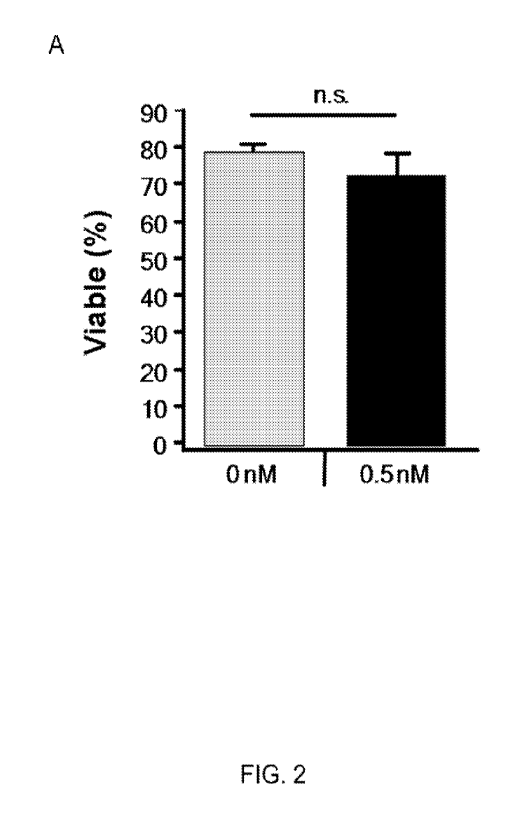

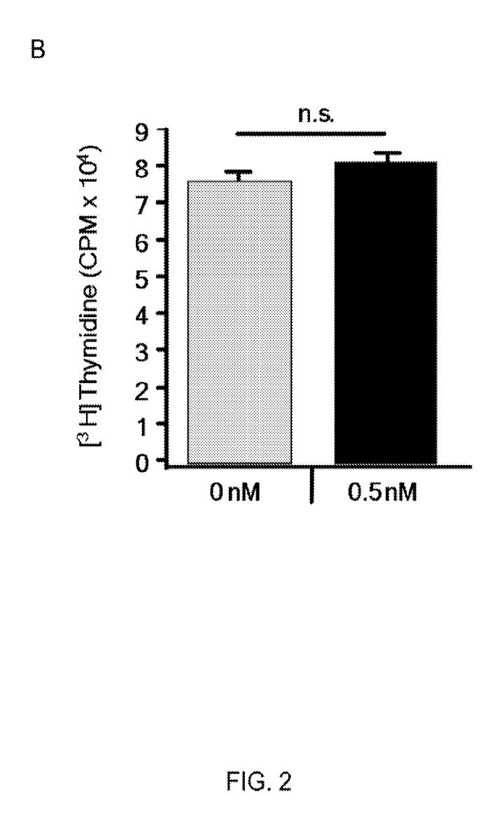

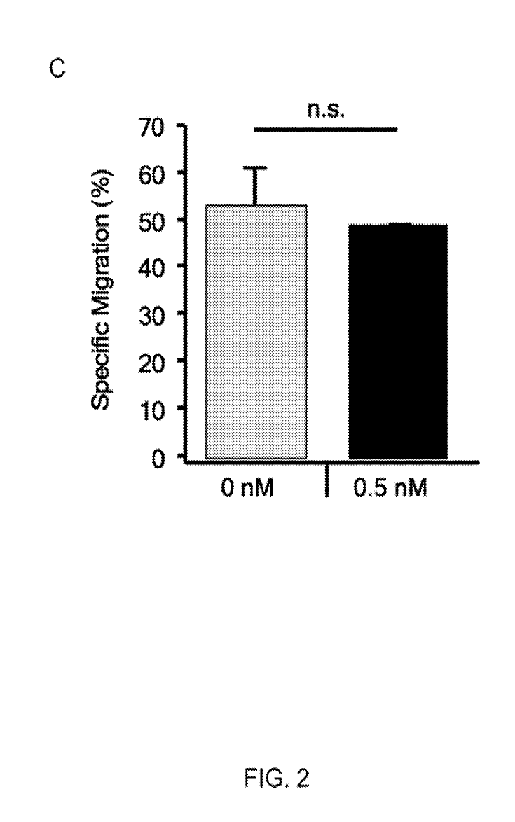

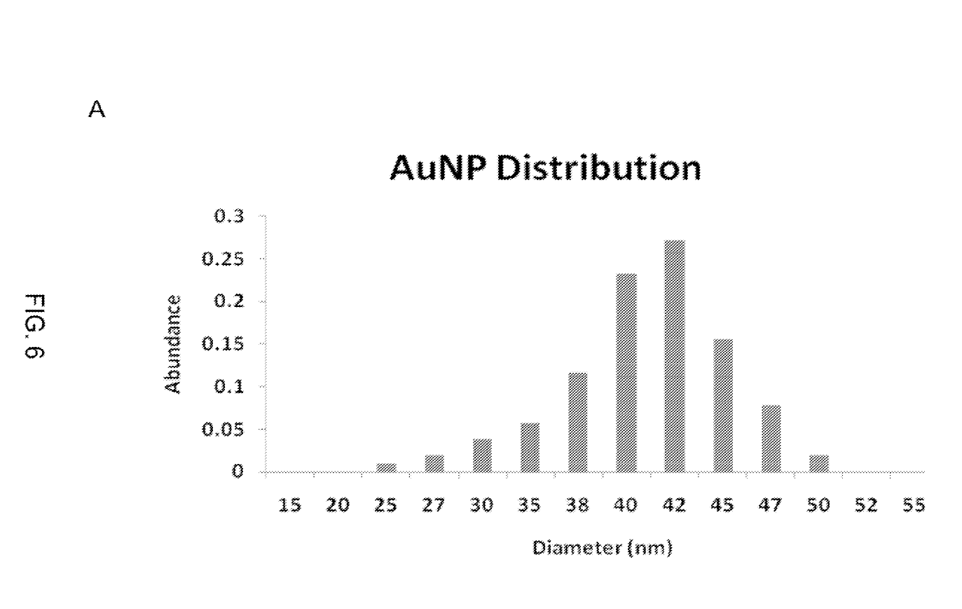

After demonstrating AuNP internalization, AuNP-T cell viability and function was assessed. Loading of T cells with AuNPs had no immediate effect on T cell viability or phenotype as determined by Annexin V/7-AAD staining (FIG. 2a, FIG. 7). Furthermore, there were no prolonged effects on T cell proliferation as measured by 24 hr [³H] thymidine incorporation (FIG. 2b). A transwell migration assay indicated that AuNP-loaded T cells were able to migrate to lymphoblastoid cell line (LCL) tumor supernatant as efficiently as non-loaded T cells (FIG. 2c), and no differences were seen in AuNP-loaded T cell IFN-□ secretion following Phorbol Myristate Acetate-Ionomycin (PMA-I) stimulation compared to non-loaded T cells (FIG. 2d). Based on these results, nanoparticle loading has no detrimental effects on T cell function and viability in vitro, indicating that in vivo migration to tumor sites will not be impaired.

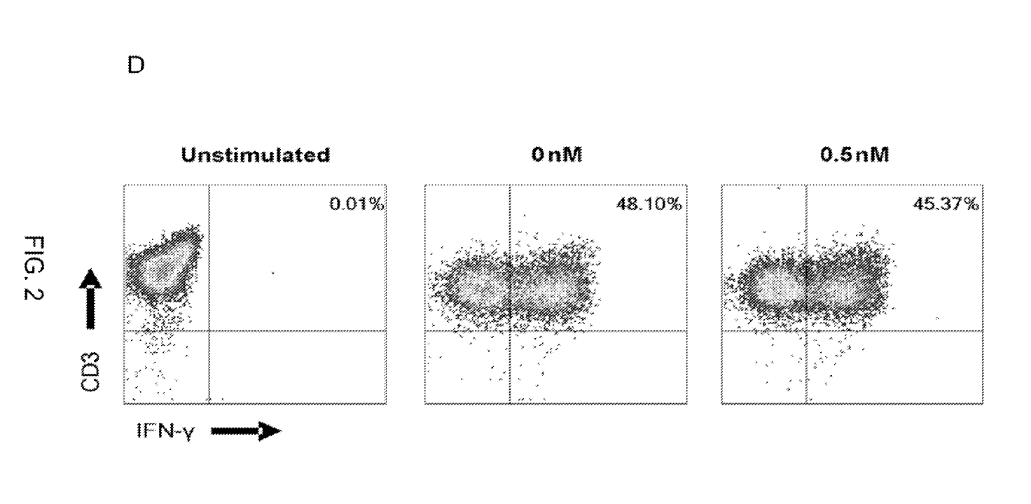

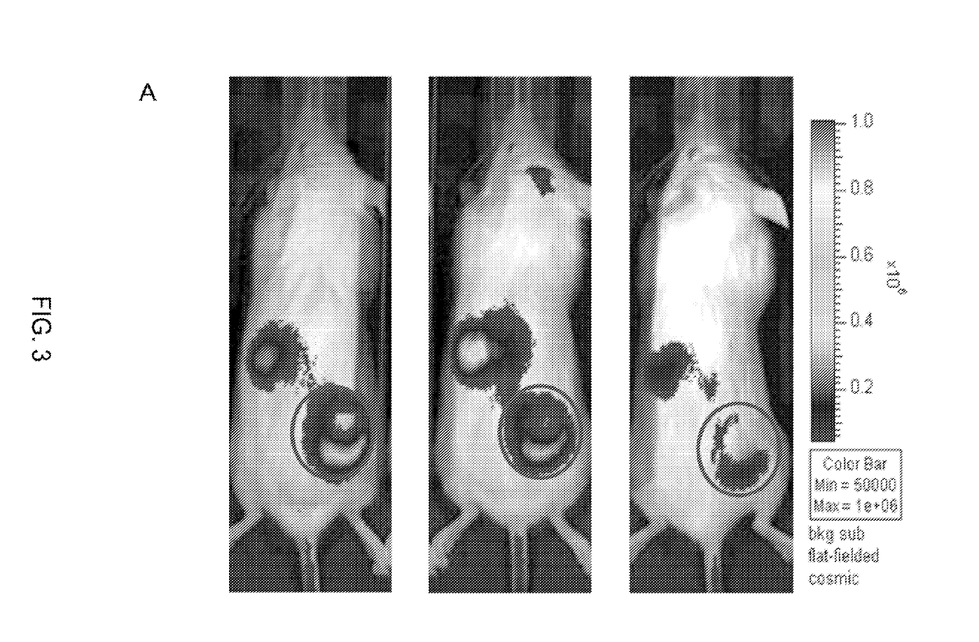

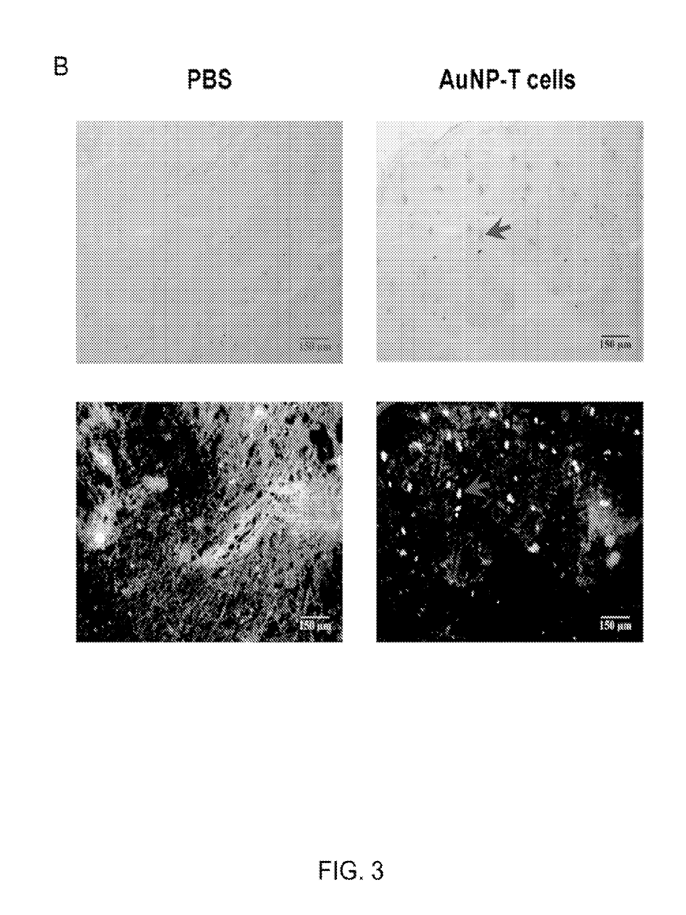

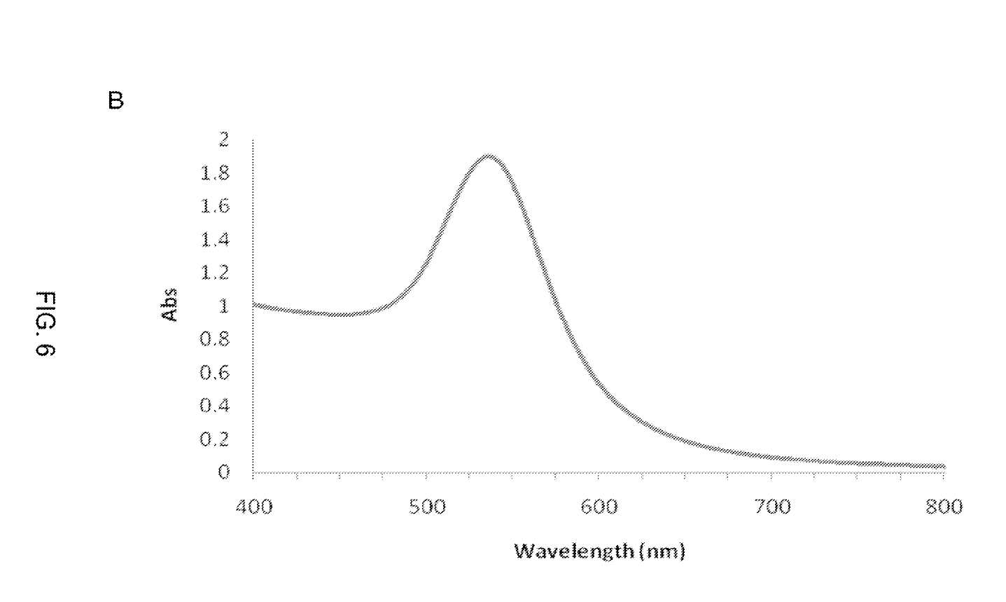

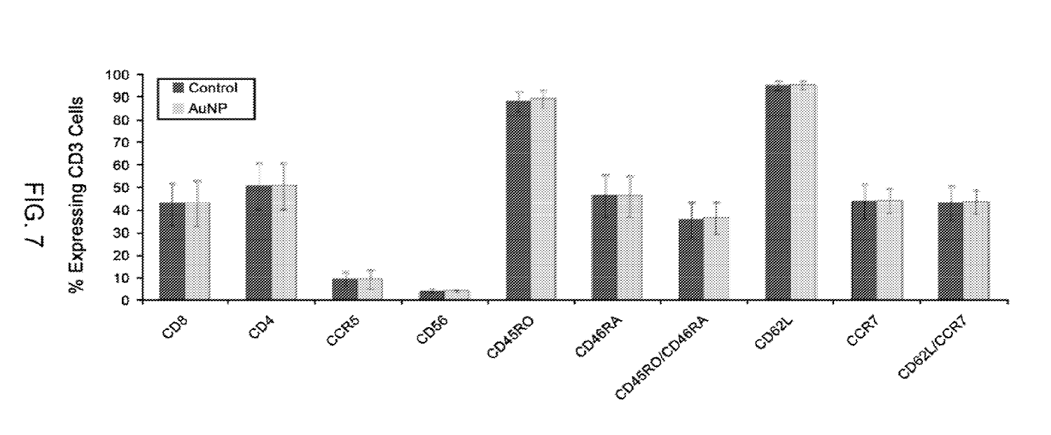

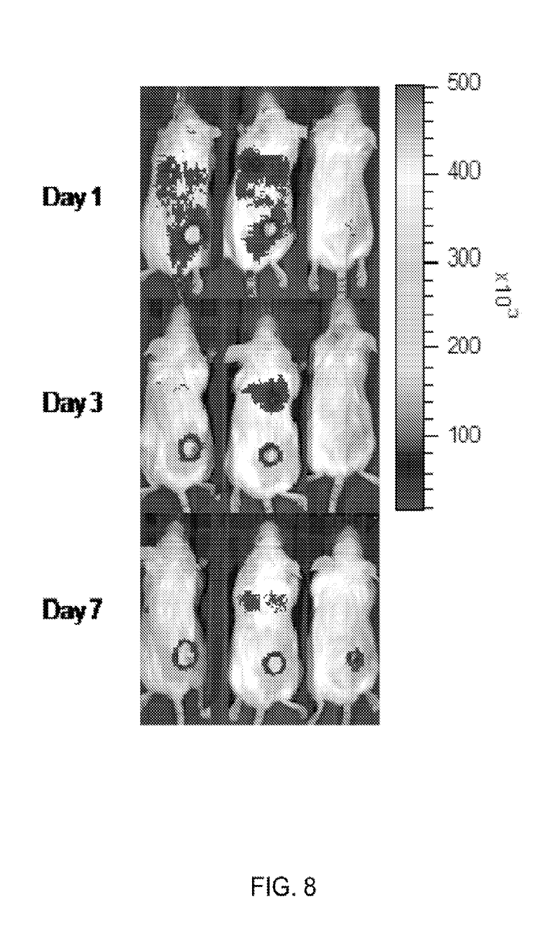

In vivo AuNP-T cell migration to the tumor was examined using bioluminescent imaging and histology (FIG. 3, FIG. 8). T cells were retrovirally modified to express firefly luciferase and were loaded with AuNPs as previously described. AuNP-T cells were systemically injected via the tail vein into LCL tumor-bearing mice. Bioluminescent imaging 48 hours postinjection confirms AuNP-T cell migration to the therapeutic site, showing accumulation within the tumor (circle) as well as spleen (FIG. 3a). Tumors were subsequently resected and cryosectioned for analysis. By immunohistochemistry the inventors detected CD3+ T cells within the AuNP-T cell treated tumors, and darkfield imaging was subsequently performed to verify the presence of AuNPs (FIG. 3d). Areas of increased scatter in the darkfield images correlate well with areas of CD3+ staining, verifying AuNP-T cell presence within the tumor.

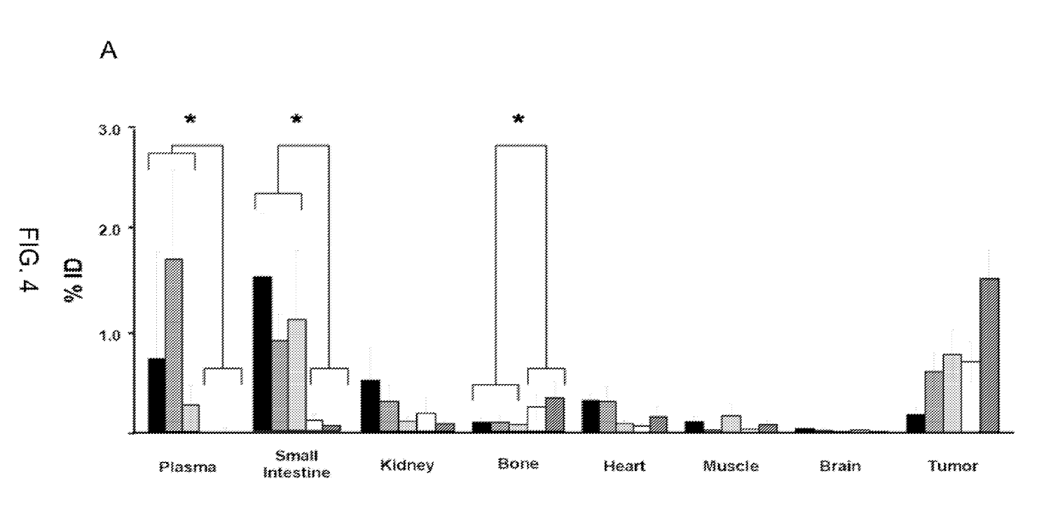

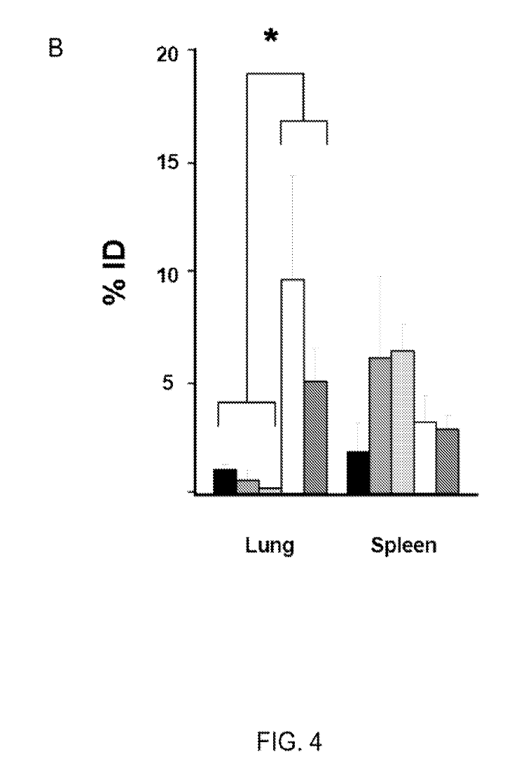

For initial comparisons of conventionally delivered AuNPs to AuNP-T cells, the inventors first performed a biodistribution time course study (FIG. 4). LCL tumor-bearing mice were administered PBS, PEG-modified gold colloid, or AuNP-T cells. Systemically-injected AuNPs were surface-coated with PEG (60-65 nm hydrodynamic diameter by dynamic light scattering) to reduce reticuloendothelial uptake. Tumors and organs (bone, brain, heart, intestine, kidney, liver, lungs, muscle, plasma, spleen) were then harvested for analysis by inductively coupled plasma mass spectrometry (ICP-MS). For AuNP-treated mice, organs were harvested at 4, 8, and 24 hours post-injection, while for AuNP-T cell treated mice, organs were harvested at 24 and 48 hours. AuNP timepoints were selected based on previous studies utilizing similarly-sized AuNPs (Perrault et al., 2009). The highest levels of tumor accumulation were seen at 24 and 48 hours for the AuNP and AuNP-T cell groups, respectively (FIG. 4a). These timepoints were chosen for delivery comparison.

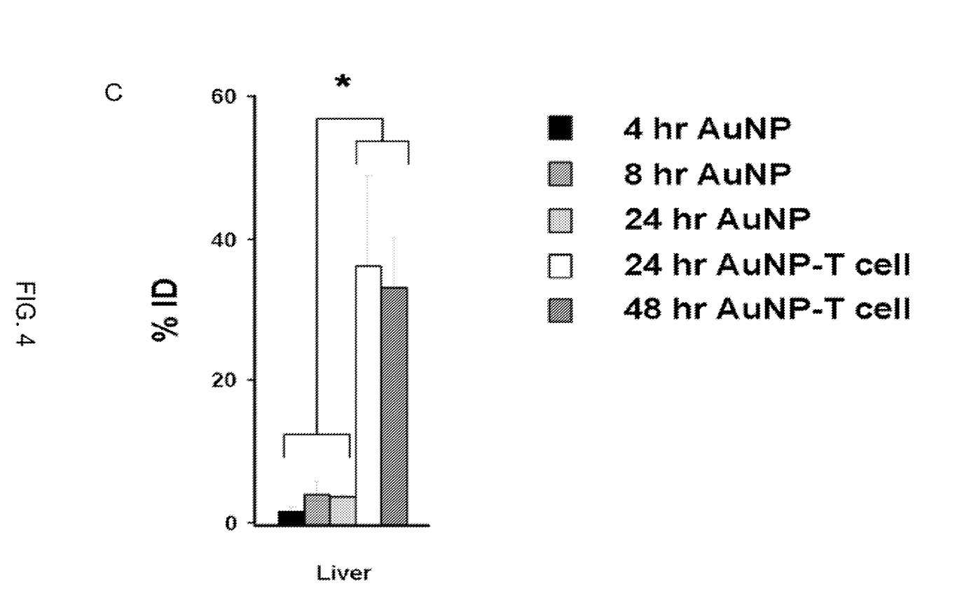

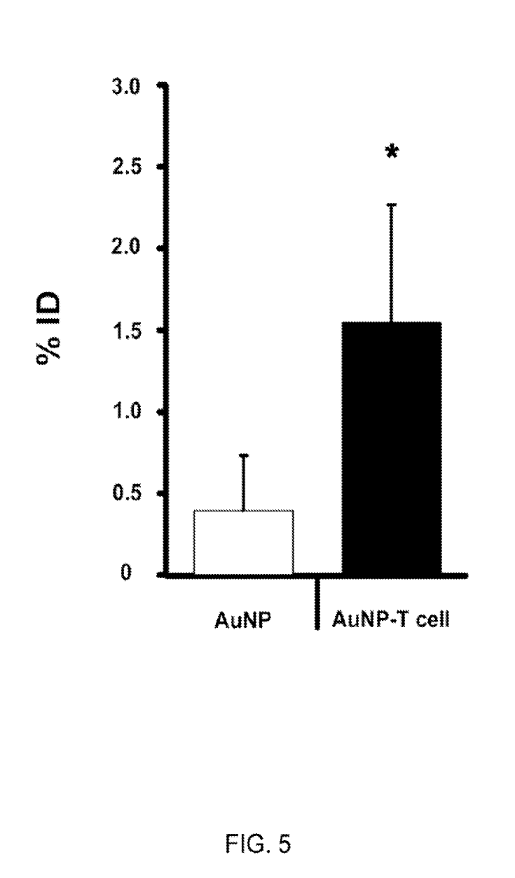

The biodistribution for the systemically injected AuNP-T cells is altered when compared to that of the gold colloid. The highest percentages of gold for the AuNP group were delivered to the liver and spleen (4.03% and 5.95% respectively at 24 hours, FIGS. 4b &4c), as has been seen in previous studies with other AuNPs (James et al., 2007; Nidome et al., 2006). This is in comparison to the AuNP-T cell group, where the highest percentages delivered were to the lung, liver, and spleen, which received 4.76%, 33.5%, and 2.69% at 48 hours, respectively (FIGS. 4b & 4c). Notably, bioluminescent imaging (FIG. 3a) shows viable AuNP-T cells of significant accumulation only in the tumor and spleen, indicating that the majority of the AuNP-T cells remaining in the lung and liver at 48 hours are dead. The AuNP-T cell biodistribution over time correlates with the normal biodistribution of human T cells seen in FIG. 8. Following systemic administration, T cells are sequestered within the capillaries of the lungs and liver. At 24 hours, T cells begin to escape the vasculature and re-enter systemic circulation, where they migrate to lymphoid organs such as the spleen and bone marrow as well as the tumor site (FIG. 4). Human T cells may be sequestered within the murine lungs and liver to a greater extent than would be expected in a syngeneic model. Nevertheless, the AuNP-T cell group delivered a higher percentage of the injected gold to the tumor than the AuNP group, with the AuNP-treated tumor receiving 0.39%±0.33% of the injected gold while the AuNP-T cell group received 1.55%±0.72% of the injected gold (p<0.01) (FIG. 5).

One of the greatest challenges of translating nanotechnologies to the clinical realm is optimizing in vivo delivery. Here, we have demonstrated enhanced AuNP delivery to tumors using human T cells as vehicles. To further enhance gold accumulation, a variety of T cell modifications could be performed, including the use of engineered tumor-specific or chemokine receptor-modified T cells. One can also optimize tumor delivery in syngeneic models, decreasing non-therapeutic site accumulation, and performing dual immuno- and AuNP-mediated therapies, in certain embodiments.

### Example 2

### Exemplary Methods for Example 1

### AuNP Synthesis and PEGylation

Gold(III) chloride trihydrate (HAuCl₄ 3H₂O 99%) and potassium carbonate anhydrous (K₂CO₃ 99%) were purchased from Sigma-Aldrich. Deionized water (18 M) was provided by a Milli-Q system. Carbon monoxide gas CO (99%) was provided by Matheson-Trigas. Au³⁺ is reduced to Au⁰ based on CO as a reducing agent. A 0.38 mM HAuCl₄ solution was prepared and aged in an amber bottle in a light protected 4° C. environment for a minimum of 72 hours prior to use. After aging the chloroauric acid solution was allowed to gradually rise to 16°. A 1.8 mM K₂CO₃ solution was prepared and allowed to age for 30 minutes prior to aeration with CO gas. The CO flow was controlled and varied via the implementation of a flow rate control valve. 40 mL of the aged solution was added to the beaker and stirred at 500 RPM prior to aeration and kept under continuous stirring. CO gas was injected into the solution at a flow rate of 30.5 mL/min. A visible color change from clear to dark purple to red is observed during synthesis, indicating formation of AuNPs. TEM images were taken to confirm size and monodispersity. Particles were sterilized by filtration through a 0.22 □m polyethersulfone filter.

To surface-coat the particles with PEG in preparation for mouse injection, 0.5 mM polyethylene glycol-thiol (PEG-SH, MW=5 kD, Nektar) was added to the particles. After a 24 hour incubation, excess PEG-SH was removed by centrifugation and PEGylated particle stability was confirmed by increasing solution tonicity with 1 M NaCl. Dynamic light scattering measurements were taken to assess the hydrodynamic diameter of the PEGylated gold colloid.

### T Cell Isolation and Preparation

Peripheral blood was obtained with informed consent from willing healthy donors using a Baylor College of Medicine Institutional Review Board approved protocol (H-15152). Peripheral blood mononuclear cells (PBMC) were isolated by Ficoll gradient centrifugation (Lymphoprep, Nycomed, Oslow, Morway). PBMC were used to generate EBV-transformed B cells lines (LCL) and T cell lines. LCL and T cells were maintained in RPMI 1640 supplemented with 10% fetal calf serum (FCS; Hyclone, Logan, Utah) and 2 mM GlutaMAX (Invitrogen, Carlsbad, Calif.).

For T cell expansion, non-tissue culture treated 24-well plates were coated with OKT3 (1 □g/mL; Ortho Pharmaceuticals, Raritan, N.J.) and anti-CD28 antibody (1 □g/mL; BD Biosciences, San Diego, Ca. overnight at 4QC. Plates were washed and 2×10⁶ PBMC were plated per well in complete RPMI supplemented with 100 U/mL recombinant human interleukin-2 (IL-2). On day 3, T cell blasts were harvested and further expanded or transduced in IL-2 supplemented media.

### T Cell Internalization of Gold Nanoparticles

Day 7 OKT3 blasts were harvested and suspended in complete RPMI supplemented with IL-2 and 0, 0.05 nM, 0.1 nM, 0.25 nM, 0.5 nM, or 1 nM of AuNPs for 24 hrs (1 mole=6.022×10²³ nanoparticles). Cells were harvested and washed extensively using 1×PBS prior to subsequent experiments.

To confirm loading, T cells were imaged using darkfield microscopy. To quantitatively characterize loading, 2×10⁶ T cells per sample were prepared for inductively coupled plasma optical emission spectroscopy (ICP-OES) analysis by digesting the cells in 3 parts trace metal grade hydrochloric acid (Fisher Scientific, Pittsburgh, Pa. and 1 part trace metal grade nitric acid (EMD Chemicals, Gibbstown, N.J.) overnight. Samples were then diluted to 10 mL in distilled water and filtered through a 0.22 □m polyethersulfone filter. T cells incubated with media alone were used as a control.

AuNP-T cells were prepared for Transmission Electron Microscopy (TEM). Cells were fixed in 4% formaldehyde in 0.1 M PBS for 1.5 hours at 4° C. The cells were rinsed in cold PBS and fixed for 1 hour in 1% OsO₄. Cells were rinsed in cold PBS then stored overnight at 4°. Cells were subsequently dehydrated using increasing concentrations of ethanol from 30-50% then stained en bloc with saturated uranyl acetate for 1 hour. Cells were infiltrated in a gradient series of ethanol and resin mixtures, then stored overnight at room temperature in 1:1 ratio of 100% ethanol:Spurr's Low Viscosity Resin. Infilitration was completed the following day with 3 changes of pure resin. Embedded cells were cured at 60° C. in 00 BEEM capsules overnight. Ultra-thin sections were cut at 70-80 nm using a Diatome diamond knife and an RMC MT6000-XL ultramicrotome. Sections were collected on 150 mesh grids and viewed on an Hitachi H-7500 transmission electron microscope (accelerating voltage=80 kV).

T Cell Viability and Functionality after Gold Nanoparticle Loading

To determine the effect of AuNP loading on T cell phenotype, the inventors used the following exemplary monoclonal antibodies conjugated to FITC, PE, PerCP or APC (BO Biosciences): CD3, CD4, CD8, CD45RA, CD45RO, CD56, CD62L, CCR5 and CCR7.

An Annexin V apoptosis detection kit (BO Biosciences) was used to determine T cell viability post-AuNP loading. Cells were analyzed using a FACSCalibur flow cytometer (BO Biosciences) and FCSExpress software (De Novo Software, Los Angeles, Cal.

A [³H] thymidine incorporation assay was used to assess the effects of AuNP loading on T cell proliferation. Following AuNP loading, T cells were seeded in triplicate into 96-well round bottom plates at 1×10⁵ cells per well in complete RPMI containing 100 U/mL IL-2 for 24 hrs. T cells were then pulsed with 5 □Ci [³H] thymidine (Amersham Pharmacia Biotech, Piscataway, N.J.) overnight. Cells were then harvested onto glass filter strips and analyzed using a TriCarb 2500 RT □-counter (Packard Biosciences, Downers Grove, Ill.).

To determine if AuNP loaded T cells retain the ability to migrate in vitro, the inventors used a transwell migration assay. T cells were labeled with 50 □Ci Chromium⁵¹ (Cr⁵¹; MP Biomedicals, Solon, Ohio) and 1.5×10⁵ cells were placed in the upper chamber of 24-well 6.5 mm diameter, 5 □m pore size transwell chambers (Costar Transwell, Corning, N.Y.). Media alone or LCL tumor supernatant was placed in the bottom chamber. Plates were then incubated for 3 hrs at 37° C. Cells in the bottom chamber were then harvested and analyzed using a □-counter Cobra Quantum, Perkin Elmer, Shelton, Conn.). Specific migration was calculated using the following equation: Specific Migration (%)=(Experimental [LCL supernatant]−Spontaneous [media alone])/(Maximum [1.5×10⁵ cells]−Spontaneous [media alone])×100.

To measure the ability of AuNP loaded T cells to secrete IFN-□ following mitogenic stimulation, 2×10⁵ T cells were seeded into 96-well round bottom plates for 24 hrs. T cells were then stimulated with 25 ng/mL Phorbol Myristate Acetate (PMA; Sigma-Aldrich, St. Louis, Mo.) and 1 □g/mL lonomycin (I; Sigma-Aldrich). Following 2 hours of PMA-I stimulation, Brefeldin A (Sigma) was added to allow for intracellular cytokine retention. 4 hrs later, cells were permeabilized using 1% Saponin (Sigma) and IFN-□ expression was detected by intracellular cytokine staining using PE-conjugated anti-IFN-□ monoclonal antibody (BD Biosciences).

### In Vivo Delivery and Therapy Studies

### SCID Xenograft Model

In vivo migration, AuNP delivery, and biodistribution studies were performed using severe combined immune deficient mice (SCID [strain ICR-Prkdc(scid)]; Taconic, Hudson, N.Y.). All mouse experiments were performed under a Baylor College of Medicine Institutional Animal Care and Use Committee (IACUC) approved protocol (BCM AN-5356/Rice A10020401). 1×10⁷ LCL tumor cells were resuspended in Matrigel (BO Biosciences) and injected subcutaneously (s.c.) into the shaved right flanks of mice. Tumors were allowed to establish and grow to at least 0.5 mm×0.5 mm in size (2-3 weeks) before use.

### Mouse Injections and Sample Collection

To prepare AuNP-T cell injections, T cells were prepared as above and incubated with 0.5 nM AuNPs for 24 hours. Cells were harvested and washed extensively using 1×PBS prior to injection. For delivery studies, mice received either phosphate buffered saline (PBS), 1×10⁷ AuNP-T cells, or 1×10¹¹ PEGylated gold nanoparticles via the tail vein in a 200 □L bolus.

To determine optimal timepoints for delivery analysis, tumors were resected at either 4, 8, or 24 hours for the PEGylated gold nanoparticle group and either 24 or 48 hours for the AuNP-T cell group. At each of these timepoints, plasma as well as portions of the liver, spleen, kidneys, small intestine, muscle, heart, lung, bone, and brain were also collected for analysis. All tissues, including tumors, were flash frozen with liquid nitrogen after collection and stored at 80° C. until analysis.

### Bioluminescent Imaging

The retroviral vector SFG.GFPluc was constructed by insertion of the GFP and firefly luciferase fusion gene (Promega, Madison, Wis.) into the SFG retroviral backbone at NcoI and MluI restriction sites. Transient retroviral supernatant was generated by co-transfection of 293T cells with the SFG.GFPluc vector plasmid, the Peg-Pam-e plasmid containing the MoMLV gag-pol sequence, and the RD114 plasmid encoding the viral envelope using GeneJuice (EMD Biosciences, Gibsstown, N.J.) transfection reagent. Viral supernatant was collected at 48 hrs and 72 hrs post-transfection.

24-well non-tissue culture treated plates were coated with 7 □g/mL Retronectin (Takara Bio, Otsu, Shiga, Japan) overnight at 4° C. The wells were washed with PBS then coated with retroviral supernatant. Day 3 OKT3 blasts were then plated at 5×10⁵ cells per well in viral supernatant supplemented with 100 U/mL IL-2. On day 3 post-transduction, cells were harvested and expanded in 24-well tissue culture treated plates in complete RPMI supplemented with 100 U/mL IL-2.

To determine if AuNP-loaded T cells can migrate to tumors in vivo and thereby deliver AuNP to the tumor site, T cells were transduced with retrovirus encoding GFPluc. Transduced cells were then loaded with AuNPs for 24 hrs then injected intravenously (Lv.) via the tail vein (1×10⁷ T cells per mouse). 48 hours post-T cell infusion, the biodistribution of T cells was visualized using the In vivo Imaging System (IVIS; Xenogen) following intraperitoneal (i.p.) injection of 150 mg/kg D-Iuciferin (Xenogen, Alameda, Calif.).

### Ex Vivo Tissue Analysis and Imaging

Resected mouse tissues were prepared and analyzed for gold content using ICP-MS and ICP-OES. Samples were lyophilized and weighed, then digested and prepared as previously described. Samples of the AuNP-T cell and gold nanoparticle boluses were also analyzed to confirm the amount of gold systemically administered.

To image AuNP-T cells within the tumor, tumors were thawed in a 37° C. water bath and embedded in optimal cutting temperature (O.C.T) compound (Sakura Finetek USA, Inc., Torrence, Calif.) using dry ice. The embedded tissue was then sectioned into 8 micron slices using a cryostat, dried overnight at room temperature, and stored at −80° C. Tissue sections were then fixed with acetone and stained for CD3 using anti-CD3 (Abcam ab5690, Cambridge, Mass.) as the primary antibody and the Invitrogen Histostain•Plus Broad Spectrum (AEC) kit. Slides were coverslipped with immunomount (Thermo Scientific, Pittsburgh, Pa.) and imaged by brightfield and darkfield microscopy.

### Example 3

### Gold Nanoparticles with Exemplary Malaria Vaccines

In specific embodiments, a vaccine for cancer treatment is employed as a pre-erythrocytic anti-malaria vaccine. Such techniques combine tumor and viral immunology with nanotechnology engineering for examination whether an exemplary novel circumsporozoite protein (CSP) nanovaccine (NV) can induce robust antigen-specific CD4+ and CD8+ T cell immunity and anti-CSP antibody production in mice.

Immunize Balb/c Mice with CSP-NV and Measure CD4+ and CD8+ T Cell Specific Immune Responses

For example, one can vaccinate Balb/c mice by subcutaneous (s.c.) injection of CSP-NV with lipopolysaccharide (LPS) adjuvant on days 0 and 15 followed by analysis of frequency of CSP-specific CD4+ and CD8+ T cells in the spleen (isolated on day 30) using an IFN-□ ELIspot assay against the CSP peptide pool and individual MHCI or MHCII-restricted epitopes.

Immunize Balb/c Mice with CSP-NV and Measure B Cell CSP-Specific Antibody Levels

Using the above vaccine strategy, one can assess levels of CSP-specific antibody production using a sandwich ELISA assay against the QGPGAP (SEQ ID NO:3) repeat motif. Here, sera samples can be taken weekly and assessed for CSP peptide-specific IgG levels.

### Background

Estimates now show between 250 and 500 million malaria cases annually worldwide, and nearly 1 million deaths in children <5 years of age (Snow et al., 2005; Who World Malaria Report 2008). Anti-malaria drugs (e.g. artemisinin-combination therapy), insecticide-treated bed nets and other vector control strategies have decreased malaria incidences in some endemic regions by up to 50% (Crompton et al., 2010; O'Meara et al., 2009), but implementation of these measures remains challenging due to poor healthcare infrastructure in developing nations and developing resistance of both the parasite and vector to drugs and insecticides, respectively. An effective vaccine that induces immunological protection may help control—and possibly eradicate—malaria when used in combination with current malaria control policies.

RTS,S represents the most promising malaria vaccine candidate. RTS,S contains the truncated circumsporozoite protein (CSP) antigen conjugated to hepatitis B surface antigens (HBsAg) (Casares et al., 2010; Gordon et al., 1995). Truncated CSP contains peptide epitopes for CD4+ and CD8+ specific T cells, as well as the NANP amino acid repeat sequence which stimulates anti-CSP specific antibodies. This single antigen pre-erythrocytic vaccine, formulated with the vaccine adjuvant AS01 (containing MPL and QS21), has demonstrated strong humoral immune responses against CSP, translating into 30-50% efficacy in clinical challenge and field studies (Casares et al., 2010).

However, the protective immunity generated by RTS,S appears to be short-lived, which may be associated with a relatively poor induction of functional (e.g. IFN-□ producing) CSP-specific CD4+ and CD8+ memory T cells to complement and sustain anti-malaria antibodies (Kester et al., 2007; Kester et al., 2009). Thus new vaccine designs or technologies aimed at stimulating robust T and B cell immune responses against CSP extends immunological protection and increases the feasibility of regional control of malaria infection, in certain aspects of the invention.

### CSP-NV: Design, Properties and Advantages

Nanoscale carriers have been attractive for vaccine and drug delivery design due to their ability to protect therapeutic payloads from degradation following administration, their improved targeting to certain tissues (e.g. vascular tumors, spleen and liver) through the enhanced permeability and retention (EPR) effect, enhanced pharmacokinetic control and cellular uptake and a large surface-to-volume ratio which increases the local concentration of the therapeutic (Maeda, 2001). A variety of nanoparticles have been used for vaccine delivery, including liposomes, viral-based nanoparticles, polymers and metallic particles.

For the vaccine, the inventors chose to use gold nanoparticles (AuNP) as a nanoscaffold to build a peptide-based malaria vaccine for several reasons. Unlike polymeric and liposomal nanoparticles (>100 nm), AuNP can be synthesized to precise size and shape specifications, which facilitates draining into lymph nodes and eventual uptake by resident professional antigen presenting cells, including dendritic cells (DC) (Kasturi et al., 2011). Indeed, nanoparticles of 45 nm or smaller show efficient lymph node draining and are taken up by immature DCs. The second advantage of AuNP is the ease of surface modification and conjugation of therapeutic molecules. Chemicals or biomolecules with thiol groups self-assemble into a monolayer on gold surfaces forming a strong Au—S bond. This process, along with being able to control the size of the AuNP core, allows better control final particle size that processes involving emulsion or encapsulation methods. Thus because of their nanoscale size, AuNP conjugated to antigen peptides, for example, provides better delivery to lymph nodes and ultimately induces stronger immune responses than other delivery systems.

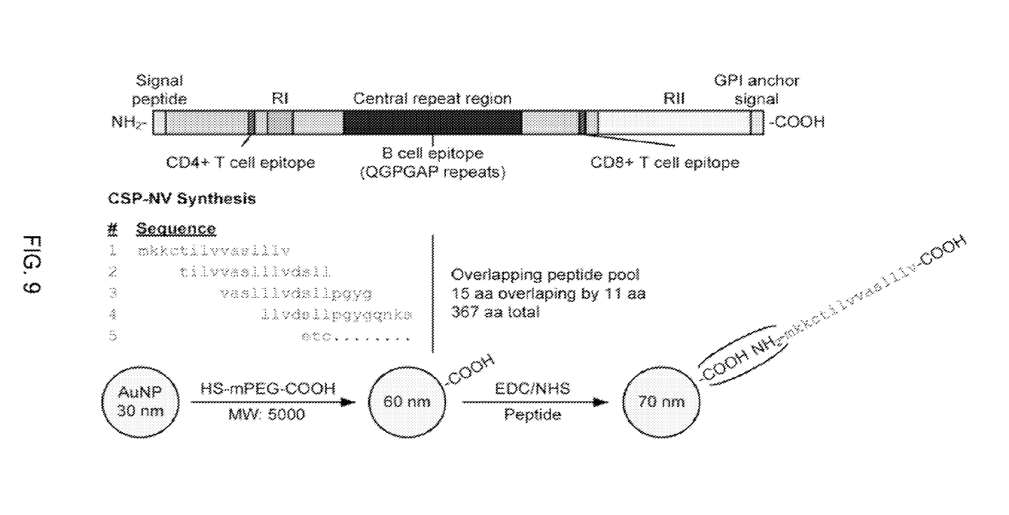

Based on a concept originally conceived for cancer, AuNP is employed as a core and as a conjugate for an overlapping peptide library of the entire CSP antigen in tandem to the nanoparticle (FIG. 9). To test this vaccine in mice, the Plasmodium yoelii CSP antigen is utilized. This design comprised of 15-mer peptides contains both CD4+ and CD8+ T cell epitopes as well as the central repeat (QGPGAP; SEQ ID NO:3) region known to stimulate antibody production.

### Exemplary Data

### Conjugation of Antigenic Peptides to AuNP

For initial studies, the inventors employed a simple yet efficient conjugation method using 1-Ethyl-3-[3-dimethylaminopropyl]carbodiimide hydrochloride (EDC), a crosslinker for carboxyl-to-amine conjugation for the attachment of antigenic peptides to AuNP. We first allowed a self-assembled monolayer (SAM) of carboxyl-PEG-thiol to form around the AuNP core overnight (FIG. 9). After aging in a 0.1 M NaCl, 10 mM sodium phosphate and 0.1% Tween-20, excessive PEG was removed by centrifugation filtration (10 k molecular weight cutoff at 2000 g for 15 minutes at room temperature (RT)). Next EDC and N-hydroxysulfosuccinimide (sulfo-NHS) linkers were added to the filtrate solution and incubated at RT for 15 minutes. Excessive linkers were subsequently removed by centrifuging the particles. Antigenic peptide(s) (e.g. gp100; H2-Db-restricted tumor antigen peptide VLYRYGSFSV; SEQ ID NO:1) were then added to the AuNP solution at 1 μg/μl and incubated overnight at RT. 10 mM of hydroxylamine was then added to quench any unbound EDC/NHS for 2 hours. The 100-coated AuNP were then washed and centrifuged three times in PBS and then resuspended in 100 μl PBS, sonicated and stored at 4° C. until use. Absorbance spectra confirmed that antigen peptides were bound to the surface of AuNP. Exemplary peptides are from the CSP protein sequence provided in GenBank® Accession No. AAA29558, incorporated by reference herein.

### Verification of Gp100 Peptide Conjugation and Immune Stimulatory Effect

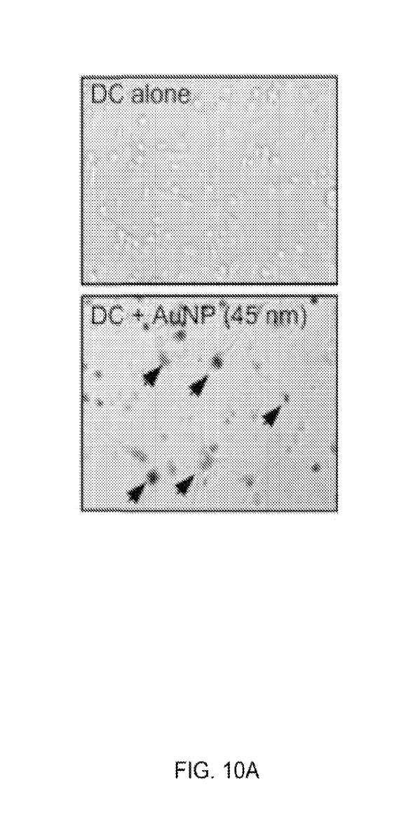

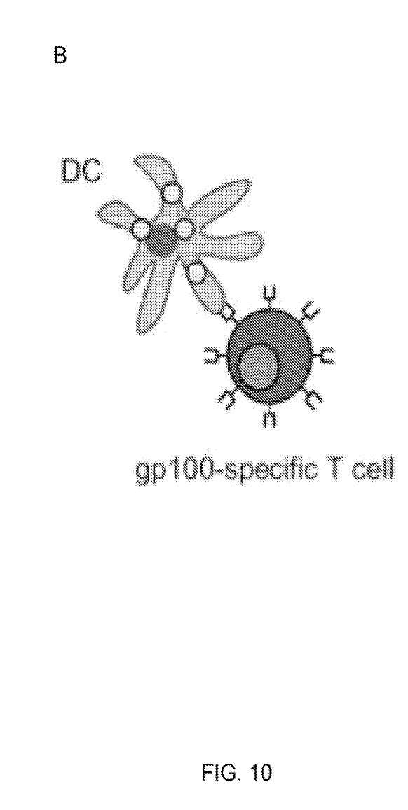

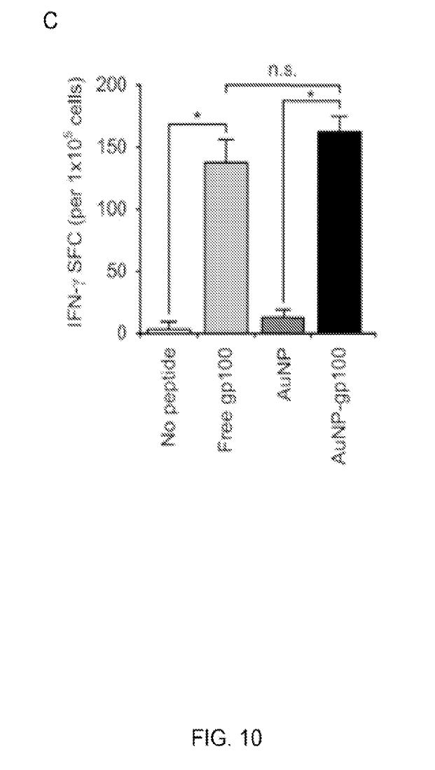

Using gp100 as a model antigen, the inventors constructed a gp100-NV particle and examined whether it could stimulate pmel-1 (gp100-specific CD8+ T cells isolated from TCR transgenic pme-1 mice) to produce IFN-□ in ELIspot assays. Using the synthesis technique described above, gp100-NV were incubated with bone marrow-derived dendritic cells which were subsequently used as APC to stimulate pmel-1 T cells. gp100-NV were efficiently taken up by DC and were capable of delivering peptide stimulation to pmel-1 responder cells (FIG. 10). This indicates that gp100 is carried into the DC and subsequently processed and presented in the context of MHCI to responding CD8+ T cells.

### Peptide Pool Synthesis for Production of Antigen-NV

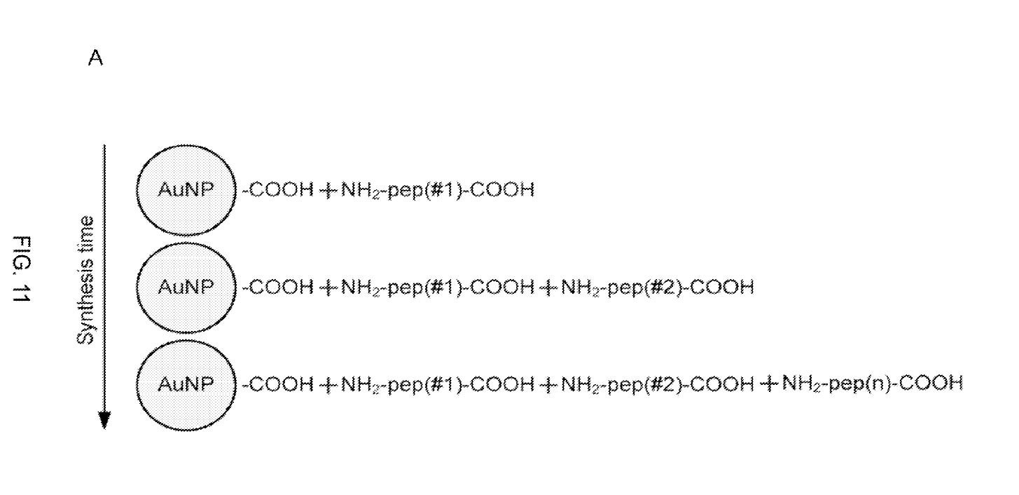

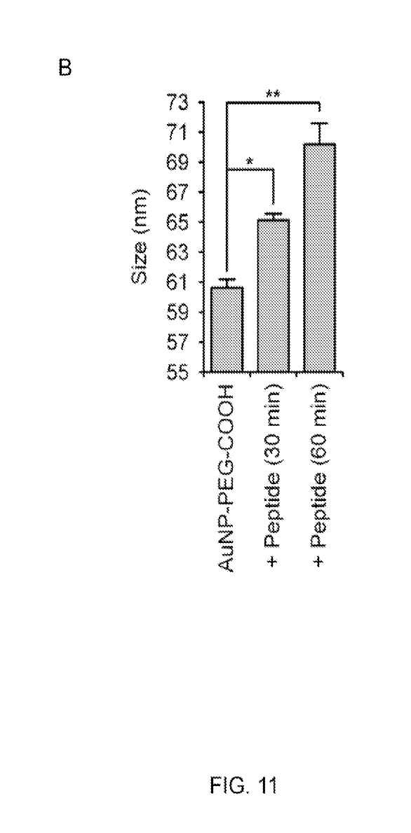

The original design was composed of a single peptide antigen. It was determined if multiple peptides could be used to make a more comprehensive nanovaccine. Here, a gp100 peptide pool (from 76 individual peptides) was conjugated to the surface of AuNP in the presence of excess linker and EDC. There was a time-dependent growth of the nanoparticle indicating at tandem linkage of peptides (FIG. 11). This indicates that each nanoparticle is bound to a variety of peptides, in certain aspects of the invention.

### Study Design

### Vaccine Strategy

Female 6- to 12-week old Balb/c mice are used to evaluate the capacity of the CSP-NV produce to induce both T and B cell immune responses. Groups of 10 mice are used for the experimental group (receiving CSP-NV), control group (no CSP-NV) and peptide alone (not conjugated to NV). Therefore for these initial studies, one can expect to use 30-40 mice. To evaluate CSP-specific T cell responses, mice are injected s.c. with 1×10¹⁴ CSP-NV resuspended in 50 □l PBS in combination with 1 □g LPS injected intraperitoneally (i.p.) on days 0 and 15. Five mice from each group are euthanized 15 days after the first immunization, and their isolated spleen cells are used for determining peptide-specific IFN-□-secreting cells using enzyme-linked immunospot (ELIspot) assays. 15 days after the second immunization, the remaining five mice are sacrificed for IFN-□ ELIspot assays. To analyze sera for the presence of CSP-specific IgG antibodies, serum is collected from peripheral blood at these same time points and analyzed as described below.

### Measurement of CSP-Specific CD4+ and CD8+ T Cell Responses Following CSP-NV Immunization

One determines the levels and breadth of the T cell response to CSP following CSP immunization, in certain aspects. In some embodiments, the generation of long-term immunological protection requires both neutralizing antibodies to CSP as well as a concurrent induction and expansion of memory CD4+ and CD8+ T cells. One can examine whether CD4+ and CD8+ T cells are generated following CSP-NV immunization.

### INF-□ ELIspot Assay

The frequency of peptide-specific T cells is determined by measuring IFN-□ secretion following stimulation with CSP peptides using ELIspot plates. Spleen cells obtained from vaccinated and control mice (five mice per group per time point) are obtained on days 0, 15 and 30 post immunization. ELIspot assays are performed using nitrocellulose microplates coated with rat capture anti-mouse IFN-□. Freshly isolated spleen cells from each mouse are plated in duplicate at concentrations (1×10⁶ and 5×10⁵ cells/ml) in RPMI containing 10% fetal calf serum (FCS). Using a peptide mixture from the CSP pool, a mixture of 10 μg of each peptide is added a final concentration of 10 ng/ml and cultured for 16 hours at 37° C. Following incubation, the plates are washed and incubated with biotinylated rat anti-mouse IFN-□ followed by incubation with streptavidin-horseradish peroxidase (HRP). The reaction is then developed using 3-amino-9-ethylcarbazole. Spots forming cells (SFC) are counted and analyzed using a third-party service (Zellnet, Inc).

### Determination of CD4+ and CD8+ T Cell Reactivity

To determine whether CD4+ or CD8+ T cells are responsible of IFN-□ production in response to CSP stimulation in our ELIspot assays, one can subsequently isolate either CD4+ or CD8+ T cells using magnetic activated cell (MACS) selection. Briefly, splenocytes are incubated with either CD4 or CD8 microbeads for 30 minutes on ice, washed and then isolated using Miltenyi MACS magnetic columns. Purified CD4+ and CD8+ T cells from vaccinated and control mice are then used as responder cells for IFN-□ ELIspot assays. Here, splenocytes from unvaccinated mice will be pulsed with 10 μg/ml CSP peptide pool for 2 hours at 37° C. to provide peptide-pulsed antigen presenting cells (APC). After washing CSP-pulsed APC are co-cultured with CD4+ or CD8+ T cells at a 1:10 ratio (APC to T cell) in ELIspot plates and developed and measured as above.

### Determining CSP Epitope Specificity

To determine the epitope specificity generated by CSP-NV immunization, one can subsequently subdivide the CSP peptide pool into sub-pools of 10 peptides each and repeat ELIspot assays to narrow potential peptide epitopes. Subsequent ELIspot assays are then performed will individual peptides to confirm specificity.

In specific embodiments, one finds elevated levels of CSP-specific CD4+ and CD8+ T cell immune responses, showing the high peak frequency at day 30, following the boosting immunization. In specific cases, s.c. injection of the CSP-NV would specifically target the LN, although in some cases i.v. injection provides a better distribution of particles and targets the spleen, as has been previously shown (Kennedy et al., 2011). If elevated levels of T cells are not observed following s.c., one can switch to i.v. injection. In addition, one can use LPS as an immune adjuvant. While this system works well in the 100-NV model in C57BL/6 mice, differences could be seen in Balb/c with the CSP antigen. Other adjuvants, such as montanide may be employed if the original approach is unsuccessful, for example.

### Measurement of Humoral Immune Response to CSP Following CSP-NV Immunization

Peptide-specific IgG levels in the sera before, during and after vaccination are measured and compared to control mice using an enzyme linked immunosorbent assay (ELISA). ELISA plates are first coated overnight at 4° C. with 1 μg/ml solution containing peptides containing the B cell epitope QGPGAP (SEQ ID NO:3) motif or irrelevant peptide control (human CMVpp65 NLVPMVATV; SEQ ID NO:2). The wells are subsequently blocked with PBS-Tween20 containing 5% bovine serum albumin for 1 hour at 37° C. Serial 1:2 dilutions (in 1% BSA-PBS-Tween20) of sera of test sera (vaccinated mice) or control sera (non-vaccinated mice) is added to the wells and incubated for 60 minutes at 37° C. Bound antibodies are then detected with alkaline phosphatase-conjugated goat anti-mouse IgG for 1 hour. Plates are then developed using phosphatase substrate tablets and measurement of the optical density at 405 nm using an ELISA plate reader. In addition to testing antibody generation to QGPGAP repeat sequences, one can also perform peptide-specific ELISA using the complete peptide pool to determine if antibodies are induce to other regions of the protein by CSP-NV.

In certain cases, one can expect to see a robust anti-CSP humoral response in these vaccinated mice. However, in some cases one can investigate using a peptide-specific ELISA assay using our QGPGAP peptides. In specific cases this is sufficient to measure CSP-specific antibodies, but if specificity is not apparent one can employ a recombinant CSP antigen for the ELISA assay, for example. In addition, one can measure total IgG to determine if CSP-NV is inducing antibodies within the mouse.

### Statistical Considerations

For the experiments described herein, one can use 10 mice per group as suggested by a biostatistician. For vaccine measurements differences between (a) IFN-□ producing cells frequency or (b) peptide-specific IgG levels are analyzed by one-way ANOVA, after log transformation if appropriate. Using a 0.025 type-I error, a sample size of 10 mice per group would provide 95% power to detect a difference in immunological output, assuming that the between-group variance is 1 and the within-group variance is 1 (log scale). Statistical significance was determined at the p<0.05 level at 95% confidence interval.

### Example 4

### Gold Nanoparticle Delivery of Exemplary miR-221 Antagomirs for the Treatment of B Cell Chronic Lymphocytic Leukemia

### Background

MicroRNAs (miRs) are small non-coding RNAs that negatively control gene expression either by regulating mRNA translation or posttranscriptional stability, or by transcriptional silencing in a sequence specific manner (Rana, 2007; Kim et al., 2008). miRs can act on many cellular functions including cell proliferation, differentiation, apoptosis and survival, and thus can act as oncogenes contributing to both initial tumorigenesis as well as resistance to chemotherapy and radiation treatment. In B-CLL, miR-221 participates directly in disease progression by deregulating entire signaling pathways that control cell proliferation and survival (Fulci et al., 2007; Fenquelli et al., 2010). As found in other malignancies, miR-221 suppresses a key regulator of cell cycle control, p27, leading to uncontrolled cell growth (Gillies et al., 2007; le et al., 2007). Importantly, miR-221 is markedly upregulated in proliferating B-CLL tumor cells found in the lymph nodes and bone marrow compared to that of non-dividing B-CLL cells found in the peripheral blood (Frenquelli et al., 2010). These data indicate that targeted inhibition of miR-221 slows tumor cell growth, in at least certain cases.

While there is clear evidence in vitro that inhibition of miRs using antisense oligonucleotides (antagomiRs) can dramatically suppress tumor growth and survival, lessons learned from clinical studies using small inhibitory RNAs (siRNA) for cancer therapy indicates that systemic administration of antagomiRs requires a delivery vehicle to minimize degradation and clearance and maximize specificity towards malignant cells, in at least certain embodiments. Recently, Davis et al demonstrated that gold nanoparticles (AuNP) could be used to efficiently deliver siRNA targeting RRM2 (M2 subunit of ribonucleotide reductase, expressed in many solid cancers) following intravenous injection in humans (Davis et al., 2010). Because of their small size (˜10-70 nm), AuNPs are rapidly taken up by a variety of cell types, including human lymphocytes (see Exemplary Data) indicating that they are useful to protect and deliver antagomiRs into B-CLL tumor cells that express miR-221. In certain embodiments of the invention, AuNP serves as a nanoscaffold to allow specific delivery of antisense oligonucleotides targeting B-CLL expressed miRs. Nanoparticle delivery of miRNA inhibitors is a novel therapeutic modality for manipulating gene expression in cancer and in other diseases.

### SPECIFIC EMBODIMENTS

### Specific Embodiment 1

Synthesize AuNPs to miR-221 locked nucleic acid (LNA) antagomiRs and test their ability to silence miR-221 using the MEC-1 B-CLL cell line expressing a reporter plasmid (see Research Plan). In addition, one can examine the effect of miR-221 inhibition on p27 protein levels as well as growth inhibition of the cell lines. Specific Embodiment 2: Evaluate miR-221 inhibition using AuNPLNAs to suppress proliferation of primary B-CLL tumor cells. Here one can stimulate primary B-CLL tumor cells with CD40L or CpG-ODNs in the presence of recombinant IL-2 (Frenquelli et al., 2010).

### Research Strategy

### Exemplary Data

Synthesis of AuNP-LNAs:

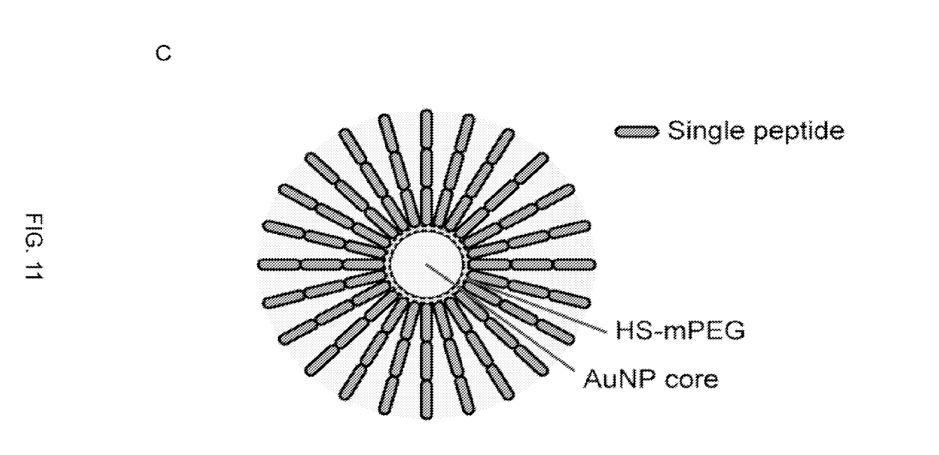

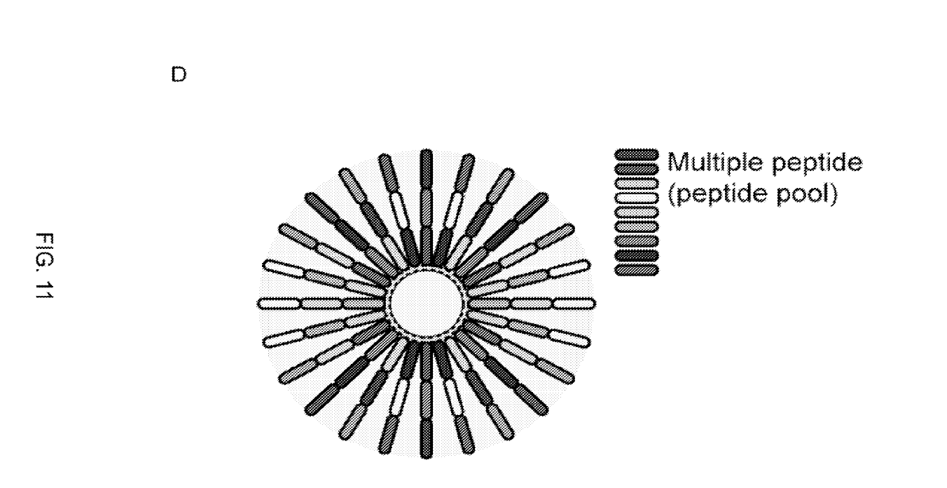

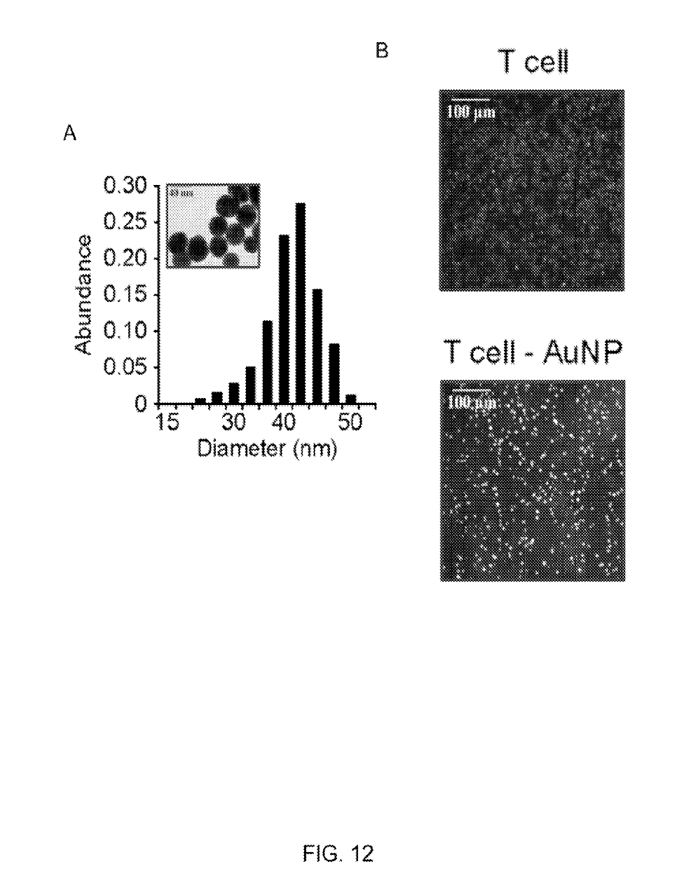

AuNPs were synthesized by carbon monoxide (CO) reduction of gold(III) chloride trihydrate (HAuCl₄ 3H₂O), producing monodispersed AuNP of approximately 40 nm in diameter (FIG. 12a). FIG. 12b demonstrates that these nanoparticles are readily taken up by T lymphocytes. To conjugate LNAs to AuNPS, LNAs targeting miR-221 are synthesized with thiol groups that allows binding to the gold surface following exposure to the reducing agent dithiothreitol (DTT). To facilitate sequestration of miRs by the AuNPLNAs, the inventors have incorporated thiol-modified oligos as spacers (FIG. 12c). Using this simple chemical bond, one can rapidly create AuNP targeting a variety of miRs.

Construction and Validation of miR-221 Inhibition Using pmiRGLO:

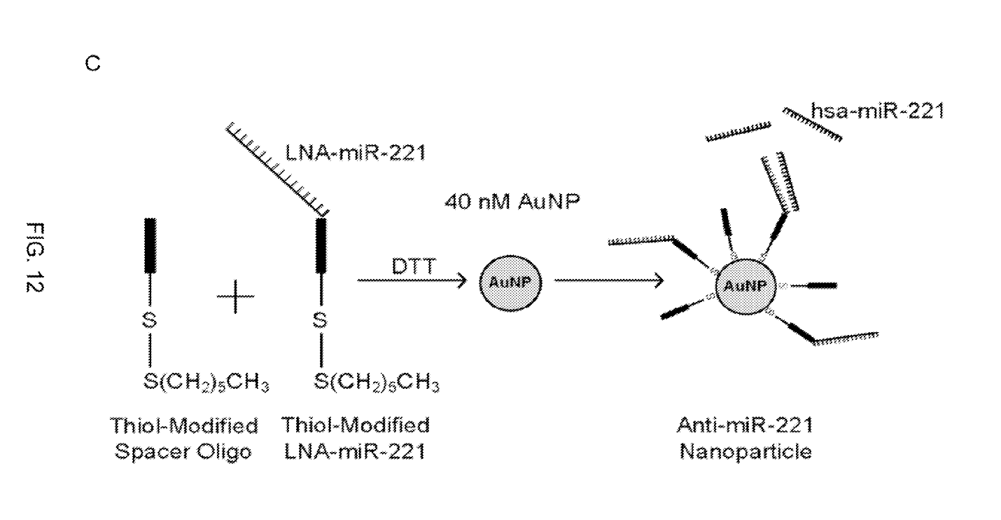

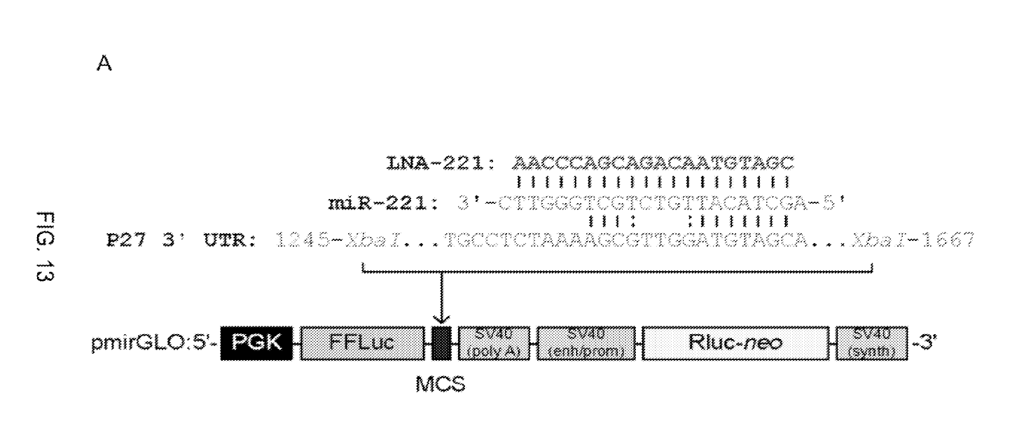

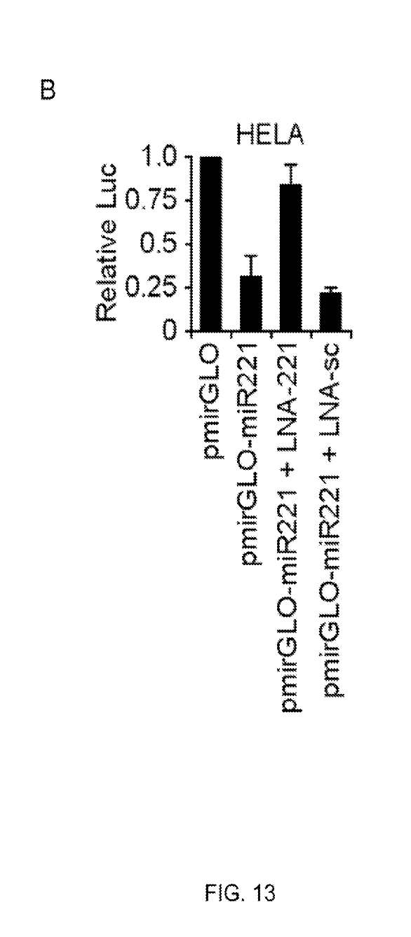

To examine whether LNAs can inhibit miRs, the inventors constructed reporter plasmid containing the 3′ UTR from p27. Here, a 422 bp fragment containing the miR-221 target sequence was cloned by PCR and inserted into the XbaI site of pmiR-GLO (FIG. 13a). Transfection of HELA cells with pmiRGLO-mi221, which express high levels of miR-221, demonstrates the negative regulation of firefly luciferase (FFluc) by miR-221 (FIG. 13b). However, transfection with LNA-221, which binds miR-221 and ablates its activity, restores expression of FFluc whereas a scrambled (non-specific) LNA does not. A second bioluminescence reporter protein (Renilla luciferase; Rluc) is used for normalization. These data indicate that LNAs targeting miR-221 can efficiently inhibit this miR.

Experimental Plan

The exemplary data and previous studies indicate that synthesis and conjugation of AuNPs to thiol-modified antisense oligonucleotides is feasible. In addition, the reporter system encoding the 3′ UTR of a primary miR-221 target gene, p2′7, functions well in HELA cells, and indicates that LNAs can efficiently knock-down miR-221. Therefore, one can test the ability of AuNP-LNA conjugates to inhibit miR-221 in the MEC-1 B-CLL tumor cell line and evaluate the effects on tumor growth.

Construction and In Vitro Validation of miR-221 Knockdown Using AuNP-LNA Conjugates

The exemplary results indicate that AuNPs with the appropriate size, monodispersity and shell chemistry can be reproducibly synthesized. In this embodiment, one can combine AuNP technology with LNA inhibition to generate a novel compound for delivery of LNAs to tumors in vivo using the chemistry described in FIG. 12, and subsequently apply it towards B-CLL. One can first synthesize LNAs (Exiqon Inc, Denmark) targeting miR-221 (5′-AACCCAGCAGACAATGTAGC-3′; SEQ ID NO:4) and a scrambled LNA (5′-GTGTAACACGTCTATACGCCCA-3′; SEQ ID NO:5) with 3′ thiol modifications for DTT binding to AuNPs. First, one can test the ability of AuNP-LNA-221 to suppress the p27 3′ UTR in our HELA cell reporter assay (FIG. 13). Next one can modify the B-CLL cell line MEC-1 (DSMZ; Germany), which expresses a high level of miR-221, with either pmiRGLO-p27 or pmiRGLO-control (containing a random 400 bp sequence) plasmids. One can then compare the efficiency of miR-221 knockdown in MEC-1 cells using AuNP-LNAs and compare luciferase expression to free LNAs. To examine whether endogenous target genes are affected by miR inhibition, one can perform Western blot analysis for p27, for example. To examine whether miR-221 knockdown by LNA or AuNP-LNAs affects MEC-1 cell growth, one can perform MTT assays (ATCC). Apoptosis of MEC-1 cells are determined by flow cytometry using annexin-V/propidium iodide staining.

### Evaluate AuNP-LNA Ability to Suppress Proliferation of Primary B-CLL Tumor Cells

Following the above embodiment, one can validate the ability of AuNP-LNA to sequester miR-221 and prevent it from suppressing p27 in HELA and MEC-1 cells, one can then examine the effects on primary B-CLL tumor cells.

Here, leukemic CD19+CD5+ B-CLL tumor cells are purified from the PBMCs obtained from B-CLL patients. B-CLL tumor cells are subsequently exposed to increasing concentrations of free LNAs (control or anti-miR-221) or AuNP-LNAs for 24 to 48 hours. B-CLL cells are then stimulated with soluble anti-CD40L 100 ng/mL CD40L+1 μg/mL enhancer; Alexis Corporation) or cytosine phosphate guanine oligodeoxynucleotide (2.5 μg/mL; stimulatory CpG-ODN type B, human specific; InVivoGen) in the presence of 100 U/ml IL-2. B-CLL proliferation following treatment with AuNP-LNAs is measured using the MTT assay (R&D Systems). One can also analyze the expression of p27 in primary B-CLL tumor cells before and after stimulation with either CD40L or CpG-ODNs, following AuNP-LNA treatment. In addition, B-CLL cells are measured for cell cycle distribution by flow cytometry using propidium iodide (PI) staining.

Thus, in this Example one characterizes whether AuNP serves as a vehicle for protecting and delivering LNA antagomiRs against miR-221 for the treatment of B-CLL. One can first test the ability of the nanoparticles to knockdown miR-221 in HELA cells and the B-CLL MEC-1 cell line. Once it is confirmed that AuNP-LNAs targeting miR-221 blocks p27 regulation, one can examine the effects on primary BCLL tumor cells. Thus, in specific embodiments, it is determined that antagomiR delivery using AuNP is useful for the targeted knockdown of miR-221 in B-CLL.

### Example 5

### pH-Sensitive Hydrazone Bonds

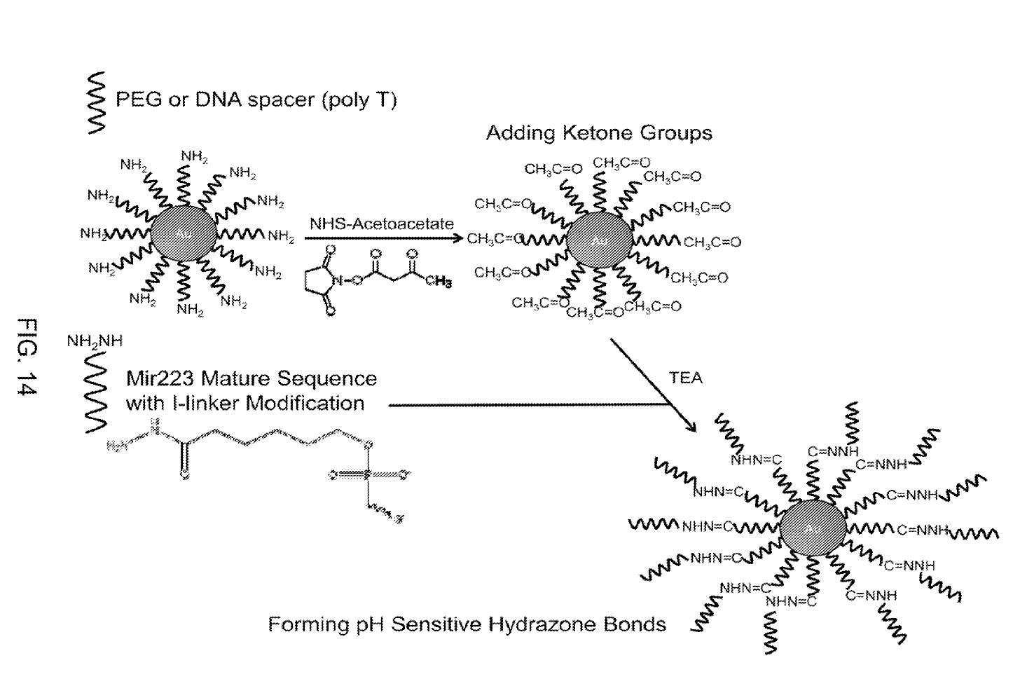

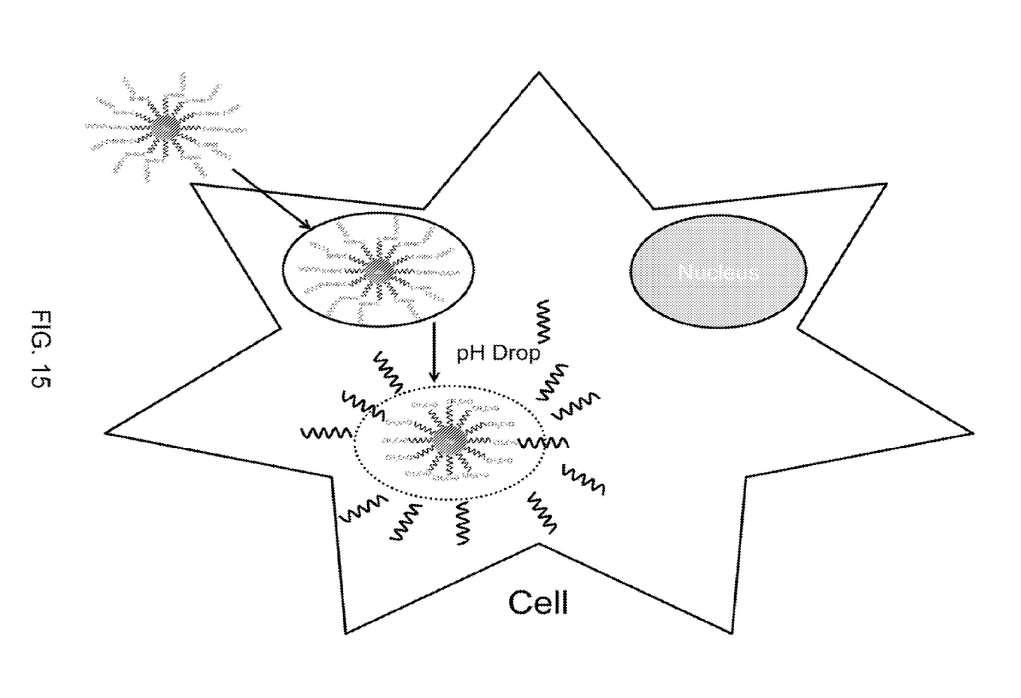

In certain embodiments of the invention, the compositions comprising the T cells and AuNP comprise pH-sensitive bonds such that upon entry into the cell, such as a cancer cell, the drop in pH results in release of the therapeutic molecule (RNA, DNA, or peptide) within the cell. This mechanism facilitates the availability of the molecules and enhances their biological function (see FIGS. 14 and 15).

In specific cases, there is pH sensitive conjugation of a nucleic acid or a peptide, for example, to a gold particle. In a particular case, miRNAs such as Mir223, mir-21, mir-138, miR-221, mir-222, mir-155, mir-181 or mir-765 (as examples only) may be conjugated in a pH-sensitive manner to the particles. Gold nanoparticles were first coated with DNA modified with thiol on one end and amine on the other. Thiols bind to gold surfaces by forming Au—S dative bonds, thus forming a self assemble monolayer. The particles were then modified with N-hydroxysuccinimidyl (NHS) acetoacetate to introduce a ketone group. pH sensitive hydrazone bonds were formed by incubating the particles with I-linker (IDTDNA) modified mature microRNA sequences.

In alternative embodiments, Thiol-PEG-amines can replace the DNA that was used to coat the gold nanoparticles for better protection from degradation. For other pH sensitive designs (such as the one for an IKK inhibitor), gold nanoparticles were first coated with DNA modified with thiol on one end and I-linker on the other. pH-sensitive hydrazone bonds were formed by incubating the particles with N-hydroxysuccinimidyl (NHS) acetoacetate modified peptides or oligos.

In alternative designs, gold nanoparticles were first coated with thiol-PEG-carboxylic acid. Hydrazine was then conjugated to the PEG particles using EDC/sulfo-NHS. pH sensitive hydrazone bonds were formed by incubating the particles with N-hydroxysuccinimidyl (NHS) acetoacetate modified peptides or oligos.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.
Casares S et al. The RTS,S malaria vaccine. Vaccine. 2010; 28:4880.
Chen, J. et al. Immuno gold nanocages with tailored optical properties for targeted photothermal destruction of cancer cells. Nano letters 7, 1318-1322 (2007).
Choi, M. R. et al. A cellular Trojan Horse for delivery of therapeutic nanoparticles into tumors. Nona letters 7, 3759-3765 (2007).
Cole, C. et al. Tumor-targeted, systemic delivery of therapeutic viral vectors using hitchhiking on antigen-specific T cells. Nature medicine 11, 1073-1081 (2005).
Cole, I R., Mirin, N. A., Knight, M. W., Goodrich, G. P., Halas, N. 1 Photothermal Efficiencies of Nanoshells and Nanorods for Clinical Therapeutic Applications. Journal of Physical Chemistry C 113, 12090-12094 (2009).
Crompton et al. Advances and challenges in malaria vaccine development. J Clin Invest. 2010; 120:4168.
Davis M E, Zuckerman J E, Choi C H, et al. Evidence of RNAi in humans from systemically administered siRNA via targeted nanoparticles. Nature. 2010; 464:1067-1070.
Di Stasi, A. et al. T lymphocytes coexpressing CCR4 and a chimeric antigen receptor targeting CD30 have improved homing and antitumor activity in a Hodgkin tumor model. Blood 113, 63926402 (2009).
Dickerson, E. B. et al. Gold nanorod assisted near-infrared plasmonic photothermal therapy (PPTT) of squamous cell carcinoma in mice. Cancer letters 269, 57-66 (2008).
Flynn, E. R. et al. Use of a SQUID array to detect T-cells with magnetic nanoparticles in determining transplant rejection. Journal of magnetism and magnetic materials 311, 429-435 (2007).
Frenquelli M, Muzio M, Scielzo C, et al. MicroRNA and proliferation control in chronic lymphocytic leukemia: functional relationship between miR-221/222 cluster and p27. Blood. 2010; 115:3949-3959.
Fulci V, Chiaretti S, Goldoni M, et al. Quantitative technologies establish a novel microRNA profile of chronic lymphocytic leukemia. Blood. 2007; 109:4944-4951.
Gillies J K, Lorimer I A. Regulation of p27Kip1 by miRNA 221/222 in glioblastoma. Cell Cycle. 2007; 6:2005-2009.
Gordon D M et al. Safety, immunogenicity, and efficacy of a recombinantly produced Plasmodium falciparum circumsporozoite protein-hepatitis B surface antigen subunit vaccine. J Infect Dis. 1995; 171:1576.
Gunn, J. et al. A multimodal targeting nanoparticle for selectively labeling T cells. Small (Weinheim an der Bergstrasse, Germany) 4, 712-715 (2008).
Harrington, K. et al. Cells as vehicles for cancer gene therapy: the missing link between targeted vectors and systemic delivery? Human gene therapy 13, 1263-1280 (2002).
Hirsch, L. R. et al. Nanoshell-mediated near-infrared thermal therapy of tumors under magnetic resonance guidance. Proceedings of the National Academy of Sciences of the United States of America 100, 13549-13554 (2003).
http://www.nanospectra.com/index.html2009).
James, W., Hirsch, L., West, J., O'Neal, P. & Payne, 1 Application of INAA to the build-up and clearance of gold nanoshells in clinical studies in mice. Journal of Radioanalytical and Nuclear Chemistry 271, 455-459 (2007).
Kasturi S P et al. Programming the magnitude and persistence of antibody responses with innate immunity. Nature. 2011; 470:543.
Kennedy et al. T cells enhance gold nanoparticle delivery to tumors in vivo. Nanoscale Research Letters. 2011; 6:283
Kershaw, M. H. et al. Redirecting migration of T cells to chemokine secreted from tumors by genetic modification with CXCR2. Human gene therapy 13, 1971-1980 (2002).
Kester K E et al. A phase I/IIa safety, immunogenicity, and efficacy bridging randomized study of a two-dose regimen of liquid and lyophilized formulations of the candidate malaria vaccine RTS, S/AS02A in malaria-naïve adults. Vaccine. 2007; 25:5359.
Kester K E et al. Randomized, double-blind, phase 2a trial of falciparum malaria vaccines RTS,S/A201B and RTS,S/AS02 in malaria-naïve adults: safety, efficacy, and immunologic associates of protection. J Infect Dis. 2009; 200:337.
Kim D H, Saetrom P, Snove O, Jr., et al. MicroRNA-directed transcriptional gene silencing in mammalian cells. Proc Natl Acad Sci USA. 2008; 105:16230-16235.
le SC, Nagel R, Egan D A, et al. Regulation of the p27(Kip1) tumor suppressor by miR-221 and miR-222 promotes cancer cell proliferation. EMBO J. 2007; 26:3699-3708.
Lu, W. et al. Targeted photothermal ablation of murine melanomas with melanocyte-stimulating hormone analog-conjugated hollow gold nanospheres. Clin Cancer Res 15, 876-886 (2009).
Maeda H. The enhanced permeability and retention (EPR) effect in tumor vasculature: the key role of tumor-selective macromolecular drug targeting. Adv Enzyme Regul 2001; 41: 189-207.
Melancon, M. P. et al. In vitro and in vivo targeting of hollow gold nanoshells directed at epidermal growth factor receptor for photothermal ablation therapy. Molecular cancer therapeutics 7, 1730-1739 (2008).
Niidome, T. et al. PEG-modified gold nanorods with a stealth character for in vivo applications. J Control Release 114, 343-347 (2006).
O'Meara et al. Changes in the burden of malaria in sub-Saharan Africa. Lancet Infect Dis. 2009; 10:545.
O'Neal, D. P., Hirsch, L. R., Halas, N. J., Payne, J. D. & West, J. L. Photothermal tumor ablation in mice using near infrared-absorbing nanoparticles. Cancer letters 209, 171-176 (2004).
Perrault, S. D., Walkey, e., Jennings, T., Fischer, H. e. & Chan, W. e. Mediating tumor targeting efficiency of nanoparticles through design. Nano letters 9, 1909-1915 (2009).

Qiao, J. et al. Purging metastases in lymphoid organs using a combination of antigen-nonspecific adoptive T cell therapy, oncolytic virotherapy and immunotherapy. Nature medicine 14, 37-44 (2008).
Rana T M. Illuminating the silence: understanding the structure and function of small RNAs. Nat Rev Mol Cell Biol. 2007; 8:23-36.
Schwartz, J. A. et al. Feasibility study of particle-assisted laser ablation of brain tumors in orthotopic canine model. Cancer research 69, 1659-1667 (2009).
Snow R W et al. The global distribution of clinical episodes of Plasmodium falciparum malaria. Nature. 2005; 434:214.
Tasciotti, E. et al. Mesoporous silicon particles as a multistage delivery system for imaging and therapeutic applications. Nature nanotechnology 3, 151-157 (2008).
Tong, L. et al. Gold Nanorods Mediate Tumor Cell Death by Compromising Membrane Integrity. Advanced materials (Deerfield Beach, Fla. 19, 3136-3141 (2007).
von Maltzahn, G. et al. Computationally guided photothermal tumor therapy using longcirculating gold nanorod antennas. Cancer research 69, 3892-3900 (2009).
WHO. WHO World Malaria Report 2008. World Health Organization Web Site. http://www.who.int/malaria/publcations/atoz/9789241563697/en/index.html.
Yotnda, P., Savoldo, B., Charlet-Berguerand, N., Rooney, C. & Brenner, M. Targeted delivery of adenoviral vectors by cytotoxic T cells. Blood 104, 2272-2280 (2004).

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of treating cancer in an individual, comprising the step of administering to the individual an effective amount of one or more compositions comprising one or more gold nanoparticles and a T cell.

2. The method of claim 1, wherein the one or more gold nanoparticles are comprised within the T cell.

3. The method of claim 1, wherein the composition localizes to a location in vivo that is accessible to heat and heat is applied thereto.

4. The method of claim 1, wherein the composition further comprises one or more peptides.

5. The method of claim 4, wherein the peptide is conjugated to the nanoparticle.

6. The method of claim 5, wherein the conjugation of the peptide to the nanoparticle is pH-sensitive.

7. The method of claim 4, wherein the peptide is from 15-40 amino acids in length.

8. The method of claim 4, wherein the peptide is further defined as an antigenic peptide.

9. The method of claim 4, wherein the one or more peptides are all the same peptides.

10. The method of claim 4, wherein the one or more peptides include different peptides.

11. The method of claim 5, wherein the peptide that is conjugated to the nanoparticle has one or more peptides attached in tandem thereto.

12. The method of claim 1, wherein the composition further comprises one or more nucleic acids.

13. The method of claim 12, wherein the nucleic acids are conjugated to the nanoparticle.

14. The method of claim 13, wherein the conjugation of the nucleic acid to the nanoparticle is pH-sensitive.

15. The method of claim 12, wherein the nucleic acid is an antisense nucleic acid.

16. The method of claim 12, wherein the nucleic acid is an antisense oligonucleotide that targets miRNA.

17. The method of claim 16, wherein the antisense oligonucleotide that targets miRNA comprises miRNA mimics.

18. The method of claim 12, wherein the nucleic acid comprises locked nucleic acid.

19. The method of claim 12, wherein the nucleic acids are no longer than 25 nucleotides in length.

20. The method of claim 16, wherein the miRNA comprises mir-221, mir223, mir155, mir-21, mir-138, mir-181 mir-122, or mir-765.

21. The method of claim 1, further comprising administering to the individual an effective amount of one or more compositions comprising one or more gold nanoparticles and a biological moiety selected from the group consisting of a peptide, a nucleic acid, and both peptide and nucleic acid.

22. The method of claim 1, wherein the T cell recognizes at least one tumor antigen.

23. The method of claim 22, wherein the tumor antigen comprises survivin, telomerase, MAGEA1, MAGEA3, MART1, RHAMM, gp100, ROR1, PRAME, SSX2, SSX4, RONIN, Her2Neu, PSMA, PSCA, or HMW-MAA.

24. The method of claim 1, wherein the T cell is modified.

25. The method of claim 24, wherein the modified T cell is further defined as being genetically modified.

26. The method of claim 25, wherein the genetically modified T cell is further defined as expressing a chemokine receptor, a tumor antigen, a chimeric antigen receptor, immune-modulating cytokines, or immune-modulating proteins.

27. The method of claim 26, wherein the chemokine receptor is selected from the group consisting of CCR7, CCR2, CXCR4, CXCR1, and CXCR2.

28. The method of claim 26, wherein the chimeric antigen receptor comprises scFv selected from the group consisting of GD2, CD19, Her2neu, PSMA, MUC1, SSEA1, and CEA.

29. The method of claim 24, wherein the modified T cell expresses CCR7, adhesion molecule CD62L, or both.

30. The method of claim 24, wherein the modified T cell is further defined as being non-genetically modified.

31. The method of claim 30, wherein the non-genetically modified T cell has been exposed to culture comprising one or more cytokines.

32. The method of claim 31, wherein the cytokine is selected from the group consisting of IL-7, IL-15, IL-12, or IL-21.

33. The method of claim 1, wherein the composition is administered to the individual more than once.

34. The method of claim 1, wherein the composition further comprises a detectable marker.

35. The method of claim 34, wherein the detectable maker comprises fluorescence, magnetism, or radioisotope.

36. The method of claim 1, wherein the shape of the nanoparticle is round, elliptical, rod-shaped, wire-shaped, prism-shaped, or is a quantum dot.

37. The method of claim 1, wherein the width of the nanoparticle is from 5 nm to 1000 nm.

38. The method of claim 37, wherein the width of the nanoparticle is from 5 nm to 200 nm.

39. The method of claim 37, wherein the width of the nanoparticle is from 40 nm to 100 nm.

40. The method of claim 1, wherein the composition further comprises polyethylene glycol (PEG).

41. The method of claim 1, wherein the composition is administered subcutaneously, intravenously, intraperitoneally, or intradermally.

42. A composition comprising one or more gold nanoparticles and a T cell.

43. The composition of claim 42, further comprising one or more peptides, one or more nucleic acids, or both.

44. A kit comprising the composition of claim 42, said composition housed in a suitable container.
